(19) United States
(12) Patent Application Publication
(10) Pub. No.: US 2007/0172846 A1
(43) Pub. Date: Jul. 26, 2007

(54) Methods for the Production and Purification of Adenoviral Vectors

(75) Inventors: Shuyuan Zhang, Sugar Land, TX (US); Hai Pham, Houston, TX (US); Ping Song, Houston, TX (US); Peter Clarke, Sugar Land, TX (US)

Correspondence Address: FULBRIGHT & JAWORSKI L.L.P., 600 CONGRESS AVE., SUITE 2400, AUSTIN, TX, 78701, US

(73) Assignee: INTROGEN THERAPEUTICS, INC., Austin, TX (US)

(21) Appl. No.: 11/559,277

(22) Filed: Nov. 13, 2006

Related U.S. Application Data
(60) Provisional application No. 60/735,614, filed on Nov. 12, 2005.
(60) Provisional application No. 60/747,960, filed on May 23, 2006.

(51) Int. Cl. C12Q 1/70; C12Q 1/68; C12N 5/00; C12N 5/02

(52) U.S. Cl. 435/6; 435/325; 435/5

(57) ABSTRACT

The present invention relates to compositions comprising and methods for producing adenovirus compositions wherein host cells are grown in a bioreactor and purified by size partitioning purification to provide purified adenovirus compositions.

This application claims priority to U.S. Provisional Applications Ser. Nos. 60/735,614 filed Nov. 12, 2005 and 60/747,960 filed May 23, 2006; and is related to U.S. patent application Ser. No. 11/187,319, filed Jul. 22, 2005; and U.S. patent application Ser. No. 11/079,986, filed May 15, 2003, all of which are hereby incorporated by reference in their entirety.

### BACKGROUND OF THE INVENTION

I. Field of the Invention

The present invention relates generally to the fields of cell culture and virus production. More particularly, it concerns improved methods for the culturing of mammalian cells, infection of those cells with adenovirus and the production of infectious adenovirus particles there from.

II. Description of Related Art

A variety of cancer and genetic diseases currently are being addressed by gene therapy. Viruses are highly efficient at nucleic acid delivery to specific cell types, while often avoiding detection by the infected host's immune system. These features make certain viruses attractive candidates as gene-delivery vehicles for use in gene therapies (Robbins and Ghivizzani, 1998; Cristiano et al., 1998). Modified adenoviruses that are replication incompetent and therefore non-pathogenic are being used as vehicles to deliver therapeutic genes for a number of metabolic and oncologic disorders. These adenoviral vectors may be particularly suitable for disorders such as cancer that would best be treated by transient therapeutic gene expression since the DNA is not integrated into the host genome and the transgene expression is limited. Adenoviral vectors may also be of significant benefit in gene replacement therapies, wherein a genetic or metabolic defect or deficiency is remedied by providing for expression of a replacement gene encoding a product that remedies the defect or deficiency.

Adenoviruses can be modified to efficiently deliver a therapeutic or reporter transgene to a variety of cell types. Recombinant adenoviruses types 2 and 5 (Ad2 and Ad5, respectively), which cause respiratory disease in humans, are among those currently being developed for gene therapy. Both Ad2 and Ad5 belong to a subclass of adenovirus that is not associated with human malignancies. Recently, the hybrid adenoviral vector AdV5/F35 has been developed and proven of great interest in gene therapies and related studies (Yotnda et al., 2001).

Recombinant adenoviruses are capable of providing extremely high levels of transgene delivery. The efficacy of this system in delivering a therapeutic transgene in vivo that complements a genetic imbalance has been demonstrated in animal models of various disorders (Watanabe, 1986; Tanzawa et al., 1980; Golasten et al., 1983; Ishibashi et al., 1993; and Ishibashi et al., 1994). Indeed, a recombinant replication defective adenovirus encoding a cDNA for the cystic fibrosis transmembrane regulator (CFTR) has been approved for use in at least two human CF clinical trials (Wilson, 1993). Hurwitz, et al., (1999) have shown the therapeutic effectiveness of adenoviral mediated gene therapy in a murine model of cancer (retinoblastoma).

As the clinical trials progress, the demand for clinical grade adenoviral vectors is increasing dramatically. The projected annual demand for a 300 patient clinical trial could reach approximately 6×10¹⁴ PFU.

Traditionally, adenoviruses are produced in commercially available tissue culture flasks or “cell factories.” Adenoviral vector production has generally been performed in culture devices that supply culture surfaces for attachment of the HEK293 cells, such as T-flasks. Virus infected cells are harvested and freeze-thawed to release the viruses from the cells in the form of crude cell lysate. The produced crude cell lysate (CCL) is then purified by double CsCl gradient ultracentrifugation. The typically reported virus yield from 100 single tray cell factories is about 6×10¹² PFU. Clearly, it becomes unfeasible to produce the required amount of virus using this traditional process. New production and purification processes that can be scaled up and validated have to be developed to meet the increasing demand.

The purification throughput of CsCl gradient ultracentrifugation is so limited that it cannot meet the demand for adenoviral vectors for gene therapy applications. Therefore, in order to achieve large scale adenoviral vector production, purification methods other than CsCl gradient ultracentrifugation have to be developed. Reports on the chromatographic purification of viruses are very limited, despite the wide application of chromatography for the purification of recombinant proteins. Size exclusion, ion exchange and affinity chromatography have been evaluated for the purification of retroviruses, tick-borne encephalitis virus, and plant viruses with varying degrees of success (Crooks, et al., 1990; Aboud et al., 1982; McGrath et al., 1978, Smith and Lee, 1978; O'Neil and Balkovic, 1993). Even less research has been done on the chromatographic purification of adenovirus. This lack of research activity may be partially attributable to the existence of the effective, albeit non-scalable, CsCl gradient ultracentrifugation purification method for adenoviruses.

Recently, Huyghe et al. (1996) reported adenoviral vector purification using ion exchange chromatography in conjunction with metal chelate affinity chromatography. Virus purity similar to that from CsCl gradient ultracentrifugation was reported. Unfortunately, only 23% of virus was recovered after the double column purification process. Process factors that contribute to this low virus recovery are the freeze/thaw step utilized by the authors to lyse cells in order to release the virus from the cells and the two column purification procedure. Of interest to the present invention is the disclosure of co-owned U.S. Published Patent Application No. 2004/0106184 A1, the disclosure of which is hereby incorporated by reference which is directed to methods for passing adenovirus particle preparations through chromatographic media to provide purified adenovirus particles.

For most of the E1 deleted first generation adenoviral vectors, production is carried out using HEK293 (human embryonal kidney cells, Invitrogen Corp.) cells which complement the adenoviral vector E1 deletion in trans. Because of the anchorage dependency of the HEK293 cells, adenoviral vector production has generally been performed in culture devices that supply culture surfaces for attachment of the HEK293 cells, such as T-flasks, multilayer Cellfactories™, and the large scale CellCube™ bioreactor system. Recently, the HEK293 cells have been adapted to suspension culture in a variety of serum free media allowing production of adenoviral vectors in suspension bioreactors. Complete medium exchange at the time of virus infection using centrifugation is difficult to perform on a large scale. In addition, the shear stress associated with medium recirculation required for external filtration devices is likely to have a detrimental effect on host cells in a protein-free medium.

Of interest to the present invention are the disclosures of co-owned U.S. Pat. No. 6,194,191 and co-owned U.S. Pat. No. 6,726,907 the disclosures of which are hereby incorporated by reference, which are directed to improved Ad-p53 production methods with cells grown in serum-free conditions, and in particular in serum-free suspension culture. Also of interest to the present invention is the disclosure of WO 00/32754 based on U.S. Ser. No. 09/203,078, the disclosure of which is hereby incorporated by reference, which is directed to the use of low-medium perfusion rates in an attached cell culture system.

Clearly, there is a demand for improved methods of adenoviral vector production that will recover a high yield of product to meet the ever increasing demand for such products. Improved methods for adenoviral vector production can include improved techniques to make production more efficient, or to optimize operating conditions to increase adenoviral vector production.

### SUMMARY OF THE INVENTION

The present invention is related to methods for producing purified viral compositions including adenovirus compositions of sufficient purity for therapeutic administration without the necessity for elaborate purification steps. Without intending to be bound by any particular theory of the invention it is believed that the steps of processing viral host cells in a cell suspension culture in a serum free media results in a viral particle product with a reduced load of contaminants. Moreover, the contaminants are of a size and nature that they may be readily separated from viral particles by a simple size partitioning purification step.

Embodiments of the invention include methods of producing purified adenovirus composition comprising one or more of steps (a), (b), (c), (d), (e), and (f), discussed in further detail below:

(a) Inoculating a bioreactor with a growth medium. The operating conditions of the cell culture may be monitored or measured by any technique known to those of skill in the art, e.g., monitoring the pH of the media and dissolved oxygen tension of the media. A growth medium can be inoculated to an initial population of host cells of at least about, at most about, or about 1×10⁴ cells/ml to about 1×10⁶ cells/ml, including any value or range of values there between. In another aspect the initial population of host cells are at a concentration of at least about, at most about, or about 1×10⁴, 5×10⁴, 1×10⁵, 5×10⁵, or 1×10⁶ cells/ml, or any value or range there between. The host cells can be capable of growing in serum-free media and are grown in a serum-free medium. According to this method, the host cells may be adapted for growth in serum-free media by a sequential decrease in the fetal bovine serum content of the growth media. Serum-free media may have a fetal bovine serum content of less than 0.03% v/v. In some embodiments, the media is CD293 media medium (Invitrogen Corp™).

The host cells may be grown at least part of the time in a perfusion chamber, a bioreactor, a flexible bed platform, or by fed batch. The cells may be grown as a cell suspension culture or alternatively as an anchorage-dependent culture. In other embodiments, media used during growth, inoculating, harvesting, and/or production phases does not contain protein and/or animal-derived products. Alternatively, host cells may be stable in serum-free and/or protein-free media.

Any cell type can be used as a host cell, as long as the cell is capable of supporting replication of an adenovirus. One of skill in the art would be familiar with the wide range of host cells that can be used in the production of adenovirus from host cells. The host cells, for example, may be 293, HEK293, PER.C6, 911, and IT293SF cells. In certain embodiments of the present invention, the host cells are HEK293 cells.

In particular embodiments, a host cell is adapted for growth in suspension culture. In particular embodiments, the cells of the present invention are designated IT293SF cells. These cells were deposited with the American Tissue Culture Collection (ATCC) in order to meet the requirements of the Budapest Treaty on the international recognition of deposits of microorganisms for the purposes of patent procedure. The cells were deposited by Dr. Shuyuan Zhang on behalf of Introgen Therapeutics, Inc. (Houston, Tex.), on Nov. 17, 1997. IT293SF cell line is derived from an adaptation of 293 cell line into serum free suspension culture as described herein. The cells may be cultured in IS 293 serum-free media (Irvine Scientific. Santa Ana, Calif.) supplemented with 100 mg/L heparin and 0.1% Puronic F-68, and are permissive to human adenovirus infection.

Any bioreactor known to those of skill in the art that is capable of supporting host cell growth is contemplated for use in the present invention. Any size of bioreactor is contemplated by the present invention, e.g., a bioreactor may be at least about, at most about, or about 10 L, 20 L up to 200 L or larger bioreactor, including any volume there between. In certain aspects a bioreactor is a bag bioreactor having a volume of at least about, at most about, or about 1, 5, 10, 20, 50, 100, 500 to 1000 L cell bag or any volume there between. A bioreactor can comprise a bioreactor that uses axial rocking of a planar platform to induce wave motions inside of the bioreactor. In some embodiments, wave motions are induced inside of a sterilized polyethylene bag wherein the host cells are located. In farther embodiments, the bioreactor is a disposable bioreactor. The bioreactor may be a commercially-available bioreactor, e.g., a Wave Bioreactor® (Wave Biotech, LLC, Bedminster, N.J.). According to one aspect of the invention a 20 L Wave Bioreactor® with an 8L working volume may be used to culture adenoviral vectors. A detailed discussion of various types of bioreactors is presented below.

(b) Growing host cells in a medium in a disposable bioreactor. Aspects of the invention include maintaining the medium at a culture temperature and the host cells grown at least about, at most about, or about 30° C., 31° C., 32° C., 33° C., 34° C., 35° C., 36° C., 37° C., 38° C., 39° C. to about 40° C., or any range derivable therein. In certain embodiments the cells are grown at 37° C. In still further aspects, the host cells can be grown at a CO₂ percentage of at least about, at most about, or about 1, 5, 10, 15 or 20%, including any percentage or range there between. Furthermore, the cells can be shaken at a speed of at least about, at most about, or about 50, 60, 70, 80, 90, 100, 110, 120, 130, 140 to 150 rpm, including any value or range there between.

(c) Providing nutrients to the host cells. Further aspects of the invention include providing nutrients to the host cells by perfusing the cells with a media containing glucose at a concentration of at least about, at most about, or about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, or 5 g/L or any concentration or range of concentration there between. The cells can be perfused at a rate to provide a glucose concentration higher than 0.5 g/L, particularly a perfusion rate of between about 0.7 and 1.7 g/L being is typically used.

The inventive methods include processing and treating the media by any method known to those of skill in the art. For example, in certain embodiments media will be perfused through a filter. The filter may be a filter that is internal to the bioreactor system, or the filter may be incorporated so that it is external to the bioreactor. In certain embodiments, the filter is a floating flat filter. The floating flat filter may be used to remove spent media from the bioreactor. Any method known to those of skill in the art may be used to monitor and maintain media volume. In some embodiments, culture volume is maintained by a load cell used to trigger fresh media addition.

In embodiments of the present invention, media may or may not be perfused into the culture of host cells. In some embodiments of the present invention, media is perfused beginning on day 3 of host cell growth. One of skill in the art would be familiar with the wide range of techniques and apparatus available for perfusing media into a cell culture system.

(d) Infecting the host cells with an adenovirus. Still further aspects of the invention include infecting the host cells at a cell density of at least about, at most about, or about 1×10⁵ to about 1×10⁷ cells/mL with an adenovirus, including all values and ranges there between. The infection temperature is typically at least about, at most about, or about 30° C., 31° C., 32° C., 33° C., 34° C., 35° C., 36° C., 37° C., 38° C., 39° C. to about 40° C., or any value or range derivable therein. In certain embodiments the infection temperature is about 37° C. The cells can be infected at an multiplicity of infection (MOI) of at least about, at most about, or about 1, 5, 10, 25, 50, 75, 100, 200, 300, 400, 500, 600, 700 to about 500, 600, 700, 800, 900, 1000 MOI, or any range or value there between, per cell. In certain aspects the host cells are infected with about 50 MOI. In further aspects the host cells are infected when at a cell density of at least about, at most about, or about 1×10⁵, 5×10⁵, 1×10⁶, 1.5×10⁶, or 1×10⁷ cells/ml, including any range or value there between. Zero to about 25, 50, 75, up to 100% of the medium may be exchanged prior to or at the time of infection. In certain aspects 100% of the medium is change at the time of infection. The growth medium can be exchange prior to or during administration of the adenovirus to the host cells.

The cells may be harvested on day 1, 2, 3, 4, 5, 6 post infection. The virus yield can be up to 2.3×10¹¹ viral particles/mL or 230,000 viral particles/cell or more. At such yields a 200 L bioreactor would be expected to yield approaching 2×10¹⁶ vp or more. In certain embodiments, the host cells are harvested following infection but prior to lysis by the adenovirus. Lysis includes, but is not limited to freeze-thaw, autolysis, or detergent lysis methods. In certain aspects cell lysis is by detergent lysis.

In embodiments of the present invention that pertain to methods of producing an adenovirus, the step of diluting host cells with fresh media may be combined with the adenovirus infection step. This is based on the inventors' discovery that these two steps can be efficiently combined to provide for excellent yields of adenoviral vectors. The invention contemplates use of any method of dilution known to those of skill in the art. In certain embodiments, the host cells are diluted 2-fold to 50-fold with fresh media and adenovirus. In other embodiments, the host cells are diluted 10-fold with fresh media and adenovirus.

In the embodiments of the present invention that pertain to methods of producing an adenovirus, the initiating of virus infection of the host cells may be accomplished by any method known to those of skill in the art. For example, in embodiments of the present invention that involve use of bioreactors, the virus infection may take place in a second bioreactor. For example, virus infection of host cells may be accomplished by adding 20-100 vp/host cell. In certain other embodiments, virus infection involves adding about 50 vp/host cell. Virus infection may be allowed to proceed for any duration of time. One of skill in the art would be familiar with techniques pertaining to monitoring the progress of virus infection. In certain embodiments of the present invention, virus infection is allowed to proceed for at least about, at most about, or about 1, 2, 3, 4, 5, 6, 7, 8, 9, 10 or more days. In certain other embodiments of the present invention, the isolating of the adenovirus from the adenovirus preparation occurs at least about, at most about, or about 1, 2, 3, 4, 5, 6, 7, 8, 9, 10 or more days after viral infection is completed.

(e) Lysing the host cells to provide a cell lysate comprising adenovirus. Yet still further aspects of the invention include lysing the host cells to provide a cell lysate comprising adenovirus using hypotonic solution, a hypertonic solution, an impinging jet, microfludization, solid shear, a detergent, liquid shear, high pressure extrusion, autolysis, sonication methods, or any combination thereof. Suitable detergents include those commercially available as Thesit®, NP-40®, Tween-20®, Brij-58®, Triton X-100® and octyl glucoside. According to one aspect of the invention the detergent is present in the lysis solution at a concentration of at least about, at most about, or about 0.5, 1, 1.5, or 2% (w/v). The concentration of contaminating nucleic acids in the crude cell lysate can be decreased by treating a lysate with a nuclease such as those available commercially as Benzonase® or Pulmozym®. In certain aspects of the present invention, the cells may be harvested and lysed ex situ. In other aspects, the cells are harvested and lysed in situ. As used herein the term “in situ” refers to the cells being located within the tissue culture apparatus, for example a CellCube™ and “ex situ” refers to the cells being removed from the tissue culture apparatus. In particular embodiments, the cells are lysed and harvested using detergent(s). In other aspects of the present invention lysis is achieved through autolysis of infected cells. The present invention also provides an adenovirus produced according to a process comprising the steps of exchanging buffer of crude cell lysate.

(f) Purifying adenovirus from the lysate. Essentially any method of isolating the adenovirus from the adenovirus preparation known to those of skill in the art is contemplated by the present invention. Aspects of the invention include purifying adenovirus from the lysate by one or more of size partitioning purification, tangential flow filtration, column chromatography, including ion exchange chromatography, such as anion exchange chromatography, or any combination thereof. In particular aspects of the invention a size partitioning membrane is in a tangential flow filtration device. In a certain aspect the size partitioning membrane is a dialysis membrane, a porous filter, or is in a tangential flow filtration device. A size partitioning membrane may have a pore size of less than about 0.001, 0.02, 0.05, or 0.08 microns and greater than about 0.0001 microns. The filtration rate can be a circulating speed of at least about, at most about, or about 500, 750, 1000 to 1000, 1250, 1500 mL/min/fsf2 and the filtration pressure is within the range of at least about, at most about, or about 0, 1, 5, 10 to 10, 20, 30 psig, or any value or range there between. In certain aspects the filtration pressure is at least about, at most about, or about 10 psig. For viruses such as adeno-associtated virus (AAV) a pore size of less than 0.01 microns but greater than 0.0001 microns is typically used.

According to one aspect of the invention, the size partitioning purification could be carried out by gel filtration purification. Such a method is not typical because gel filtration size partitioning effects a dramatic increase in volume and dilutes the viral preparation. Such diluted preparations must then be reconcentrated which is typically costly and undesirable.

In other aspects of the invention a virus may be purified to a pharmaceutically acceptable degree without the use of additional purification steps such as ion exchange chromatography. By pharmaceutically acceptable degree is meant substantially free of animal derived components and free of other protein impurities as seen on an SDS-PAGE gel so as to not impact on the human clinical use of the product.

The methods may also include concentrating and diafilitering the lysate. Diafiltration can be by tangential flow filtration. In certain aspects the membrane capacity is at least about, at most about, or about 2 L/1.1 ft² to about 6 L/1.1 ft², including all values and ranges there between. In a further aspect the concentration fold may be in the range of at least about, at most about, or about 5-fold, 10-fold, 15-fold to 20-fold, or more, including any value or range there between. The feeding flow rate may be in the range of at least about, at most about, or about 500, 600, 700, 800, 900, 1000, 1100, 1200, 1300, 1400 or 1500 ml/min, or any range or value there between. Typically the purified adenovirus has a purity of less than 10, 5, 1, 0.5, or 0.1 nanograms of contaminating DNA per 1 milliliter dose. In certain embodiments a composition will comprise at least about, at most about, or about 1×10¹², 5×10¹², 1×10¹³, 5×10¹³, 1×10¹⁴, 5×10¹⁴, 1×10¹⁵, 5×10¹⁵, 1×10¹⁶, 5×10¹⁶ or 1×10¹⁷ viral particles, including all values there between. Typically the viral particles are obtained from a single culture preparation. In particular embodiments, the methods comprise a concentration step employing membrane filtration. Membrane filtration may utilize a 100 to 1000K NMWC, regenerated cellulose, or polyether sulfone membrane.

The ability to produce purified adenoviral preparations without traditional chromatographic purification steps provides significant improvements in viral production yields while reducing expense. Specifically, the invention provides a method for removing contaminants from a virus-containing composition comprising obtaining an aqueous composition comprising a selected virus and undesirable contaminants, and subjecting the aqueous composition to size partitioning purification using a size partitioning membrane having partitioning pores that retain virus and permit the passage of contaminants to remove contaminants and provide a purified virus composition. Of course, the size of the partitioning pores can be selected on the basis of the size of the virus to be retained, in which case one will select a membrane having a pore or inclusion size sufficiently smaller than the virus to retain the virus and permit the passage of contaminants. Similarly, if the pore or inclusion size is too small, some undesirable contaminants may be retained. Therefore, an optimal pore size is one that retains the most virus yet permits the passage of the most contaminants. Generally, the size of the virus and corresponding proposed pore sizes will be as in Table 1 below:

TABLE 1

| Virus | Average Particle Size | Pore Size Range |
|---|---|---|
| Adenovirus | 80 nm | Ÿ 0.05 ÿm |
| AAV | 20 nm | Ÿ 0.01 ÿm |
| Retroviruses | 100 nm | Ÿ 0.05 ÿm |
| Herpes virus | 100 nm | Ÿ 0.05 ÿm |
| Lentivirus | 100 nm | Ÿ 0.05 ÿm |

Some embodiments of the present invention involve analysis of virus production. For example, virus production may be analyzed using HPLC. Any technique for analyzing virus production known to those of skill is contemplated by the present invention.

The methods of the invention may be used when the virus is adenovirus, lentivirus, adeno-associated virus, retrovirus or herpes virus. According to one aspect of the invention the viral particles are intended for use in gene therapy or vaccination. Accordingly, the viral particle is an adenovirus which comprises an adenoviral vector encoding an exogenous gene construct. A recombinant or exogenous gene can be operatively linked to a promoter. Any promoter known to those of skill in the art can be used, as long as the promoter is capable of functioning as a promoter. For example, in certain embodiments the promoter is an SV40 EI, RSV LTR, β-actin, CMV-IE, adenovirus major late, polyoma F9-1, or tyrosinase promoter.

In embodiments of the present invention where the adenovirus is an adenovirus encoding a therapeutic gene, an exogenous gene, and/or a recombinant gene, any recombinant gene, particularly a therapeutic gene, is contemplated by the present invention. For example, the recombinant, exogenous, or therapeutic gene can be, but is not limited to antisense ras, antisense myc, antisense raf, antisense erb, antisense src, antisense fms, antisense jun, antisense trk, antisense ret, antisense gsp, antisense hst, antisense bcl, antisense abl, Rb, CFTR, p16, p21, p27, p57, p73, C-CAM, APC, CTS-1, zacl, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus-1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zacl, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC. In further embodiments of the present invention, the recombinant gene is a gene encoding an ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. In other embodiments of the present invention, the recombinant gene is a gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase. Alternatively, the recombinant gene may encode growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

Viral vectors include adenoviral vectors and particularly those in which the adenovirus is a replication-incompetent adenovirus. Such replication incompetent adenoviral vectors include those in which the adenovirus is lacking at least a portion of the E1-region with those lacking at least a portion of the E1A and/or E1B region being typical. A replication incompetent adenovirus can be produced in host cells which are capable of complementing replication. The inventive processes offers not only scalability and validatability, but also excellent virus purity.

In some embodiments of the invention, the adenovirus that is isolated is formulated in a pharmaceutically acceptable composition. One of skill in the art would be familiar with the extensive methods and techniques employed in preparing pharmaceutically acceptable compositions. Any pharmaceutical composition into which adenovirus can be formulated is contemplated by the present invention. For example, certain embodiments of the invention pertain to pharmaceutical preparation of adenovirus for oral administration, topical administration, or intravenous administration.

In some embodiments of the invention, the methods for producing an adenovirus disclosed above and elsewhere in this specification concern methods for isolating and purifying an adenovirus that involve obtaining a purified adenovirus composition having one or more of the following properties: (1) a virus titer of at least about, at most about, or about 1×10⁹, 1×10¹⁰, 1×10¹¹, 1×10¹² to at least about, at most about, or about 1×10¹³, 1×10¹⁴, 1×10¹⁵ pfu/ml; (2) a virus particle concentration of at least about, at most about, or about 1×10¹⁰, 1×10¹¹ to at least about, at most about, or about 2×10¹³ 1×10¹⁴, 1×10¹⁵ particles/ml; (3) a particle:pfu ratio at least about, at most about, or about 10, 20, 30, 40, 50 to at least about, at most about, or about 60; (4) having less than 50, 40, 30, 20, 10, 5 ng BSA per 1×10¹² viral particles; (5) at least about, at most about, or about 50, 40, 30, 20, 10 pg and 1 ng of contaminating human DNA per 1×10¹² viral particles; (6) a single HPLC elution peak consisting essentially of 97%, 98%, 99% to 100% of the area under the peak. In certain embodiments, the adenovirus composition prepared in accordance with the steps discussed above includes at least about, at most about, or about 5×10¹⁴, 5×10¹⁵, 5×10¹⁶, 5×10¹⁷, and 1×10¹⁸ viral particles, or any value or range there between.

A virus may be formulated as composition for administration to a subject for a variety of uses, such as cancer therapy or vaccination. Furthermore such formulation may be designed for storage at refrigerated temperatures or room temperature. Significant reductions in virus particle concentration and infectivity have been observed when a virus is present in oxidating conditions. Therefore, the present invention provides various formulation that contain anti-oxidation excipients. Based on the inventors' experience and literature reference alpha tocopherol; ascorbic acid; glutathione; sucrose, fructose; galactose; lactose; maltose and other sugars; ethanol; glucose; ascorbyl palmitate; ascorbyl stearate; anoxomer; butylated hydroxyanisole; butylated hydroxytoluene; citric acid; citrates; erythorbic acid and Na erythorbate; ethoxyquin; ethylenediaminetetraacetic acid; Ca disodium salt; propyl, octyl, dodecyl gallates; glycine; gum guaiac; ionox 100; (2,6-di-tert-butyl-4-hydroxymethylphenol); lecithin; polyphosphates; tartaric acid; tertiary butyl hydroquinone; trihydroxy butyrophenone; thiodipropionic acid; diauryl and distearyl esters; and arginine can be used as anti-oxidation agents in adenovirus formulations. An anti-oxidant excipient may be present at least about, at most about, or about 0.01, 0.05, 0.1, 0.5, 1, 2, 3, 4, 5, 6, 7, 8, 9, 10% v/v or w/v of an adenoviral formulation. In certain embodiments different concentrations of ethanol can be added to an adenovirus vector preparation with a virus particle concentration of, for example, 1.2×10¹² vp/mL or more. Typically, ethanol protection is concentration dependent. Protection against oxidation may be affected at concentrations as low as 0.5% v/v. Ethanol may be a component of a liquid formulation in concentration of at least about, at most about, or about 0.5, 1, 2, 3, 4, 5, 6, 7, 8, 9, 10% v/v or more and any percentage there between, of the adenoviral formulation. Overall the data indicate that ethanol is an effective anti-oxidant that could be used to stabilize adenoviral formulations.

In other aspects of the invention the amino acid Arginine can be used as excipient for the formulation of adenovirus. Because of the presence of an unsaturated bond in the Arginine molecule, it may be considered an anti-oxidant. Similar studies to those described herein for ethanol were carried out using Arginine. Protection was concentration dependent. Protection was seen at 1 and 10 mM concentrations. Arginine may be a component of a viral composition and be present in concentration of at least about, at most about, or about 0.5, 1, 2, 3, 4, 5, 6, 7, 8, 9, 10, 11, 12, 13, 14, 15 mM or more.

Ethanol and arginine may be included in a base formulation that includes, but is not limited to at least about, at most about, or about 0.5, 1, 5, 10, 15, to at least about, at most about, or about 20 mM Tris; and/or at least about, at most about, or about 0.05, 0.1, 0.15, 0.25, to 0.5 M NaCl; and/or at least about, at most about, or about 0.01, 0.05, 0.1, 0.2, 0.5, to 1% Tween-80; and/or at least about, at most about, or about 0.01, 0.05, 0.1, 0.5, 0.75, to 1% PEG; and/or at least about, at most about, or about 0.01, 0.1, 0.5, 1, 5, 10, to 20% sucrose or glycerol, including all values and ranges there between; at a pH of at least about, at most about, or about 7.0, 7.5, 7.6, 7.7, 7.8, 7.9, 8.0, 8.1, 8.2, 8.3, 8.4, 8.5, 8.6, 8.7, 8.8, 8.9, to about 9.0. Adenovirus can be formulated in the inventive formulations of at least about, at most about, or about 1×10⁵, 1×10¹⁰ 1×10¹¹, 2.5×10¹¹, 5×10¹¹, 1×10¹², 2.5×10¹², 5×10¹², 1×10¹², 2.5×10¹³, 5×10¹³, 1×10¹⁴, 2.5×10¹⁴, 5×10¹⁴, 1×10¹⁵, 2.5×10¹⁵, 5×10¹⁵ vp/mL or higher concentrations, including all concentrations or ranges of concentration there between. The formulated virus may be stored at least about, at most about, or about 4, 5, 6, 7, 8, 9, 10, 15, 20, 25° C. and/or room temperature, which is typically 20 to 25°, for extended period of time, e.g., 5, 10, 15, 20, 25, 30 days, weeks, or months and may include 1, 2, 3, 4, 5, 6 or more years.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Effect of cell seeding density on cell growth

FIG. 2 Effect of temperature on cell growth.

FIG. 3 Growth curve related to CO₂ percentage.

FIG. 4 Cell growth and viability in Wave bioreactor (cell line A)

FIG. 5 Cell growth and viability in Wave bioreactor (cell line B)

FIG. 6 Volumetric virus yield at harvest.

FIG. 7 Specific virus yield at harvest.

FIG. 8 Volumetric virus yield at harvest.

FIG. 9 Specific virus yield at harvest.

FIG. 10 Volumetric virus yield at harvest

FIG. 11 Specific virus yield at harvest.

FIG. 12 Volumetric virus yields at harvest.

FIG. 13 Specific virus yield at harvest.

FIG. 14 Effect of filtration flow rate and pressure on virus titer

FIG. 15 Effect of filtration rate and pressure on virus titer.

FIG. 16 Volumetric Processing Capacity of UF/DF membrane (1.1 ft²).

FIG. 17 UF/DF concentration fold using 1.1 ft² membrane.

FIG. 18 Processing flow rate of UF/DF Membrane (1.1 ft²)

FIG. 19 Endonuclease digestion assay.

FIG. 20 Effect of H₂O₂ on virus concentration

FIG. 21 Effect of H₂O₂ on virus infectivity.

FIG. 22 Effect of ethanol on protecting adenovirus against oxidation by H₂O₂.

FIG. 23 Effect of ethanol on protecting adenovirus against oxidation by H₂O₂.

FIG. 24 Effect of arginine on protecting adenovirus against H₂O₂ oxidation.

FIG. 25 Purification scheme.

FIG. 26 PFD for down stream processing and purification.

FIG. 27 PFD for bulk drug product formation.

FIG. 28 PFD for fill of drug product.

### DESCRIPTION OF ILLUSTRATIVE EMBODIMENTS

It has been shown that adenoviral vectors can successfully be used in eukaryotic gene expression and vaccine development. Recently, animal studies have demonstrated that recombinant adenovirus could be used for gene therapy. Successful studies in administering recombinant adenovirus to different tissues have proven the effectiveness of adenoviral vectors in therapy. This success has led to the use of such vectors in human clinical trials. There now is an increased demand for the production of adenoviral vectors to be used in various therapies. The techniques currently available are insufficient to meet such a demand. The present invention provides methods for the production of large amounts of adenovirus for use in such therapies and the formulation of adenovirus for prolonged periods of time, in certain aspects at refrigerated or room temperatures.

Therefore, the present invention is designed to take advantage of improvements in large scale culturing systems and purification for the purpose of producing and purifying adenoviral vectors. The various components for such a system, and methods of producing adenovirus are set forth below.

I. Virus Production and Processing

Aspects of the invention include the characterization and optimization of the adenovirus vector production process using a suspension process, particularly the “Wave” process, and chromatography purification. Exemplary methods can be found in U.S. Pat. Nos. 7,125,706, 6,726,907, 6,689,600, and 6,194191, and U.S. Patent publications 20060166364, 20050089999, 20050158283, 20040229335, 20040106184, 20030232035, 20330229354 20020182723, and 20020031527, each of which is incorporated herein by reference in its entirety. Exemplary materials include 293 suspension cells, which may be engineered to express adenovirus or other therapeutic viruses; HeLa suspension cells; Media, in some instances CD-293 (Invitrogen Formulation # 03-0094DK) or other appropriate medias that are readily available to one skill in the art; Erlenmeyer flasks (Coming 431145); bioreactor, in certain aspects a Wave bioreactor or other similar bioreactors. Cell concentration and viability determination were determined in part by staining with trypan blue and counting using a hemacytometer under a microscope.

A. Upstream Cell Culture and Adenovirus Amplification

Cell seeding density. Host cell suspension stocks, such as 293 suspension cell stock, may be used to seed shaker flask, bioreactor or other cultures at various seeding densities. Satisfactory cell growth may be achieved with a wide range of cell seeding densities. A longer lag phase may be associated with cell seeding densities lower than 1×10⁵ cells/mL. For optimal cell growth the cell seeding density is recommended to be at least about, at most about, about, or higher than 1×10⁵ cells/mL and includes, but is not limited to cell densities of at least about, at most about, or about 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶ cells/mL, including all values or ranges there between.

Culture temperature. Cells can be cultured at temperatures that include, but are not limited to at least about, at most about, or about 32° C., 33° C., 34° C., 35° C., 36° C., 37° C., 38° C., 39° C. or 40° C., including all values there between. In certain aspects of the invention the incubation temperature for growth of 293 suspension cells will be at no less than 35° C. and typically at 37° C.

CO₂ percentage. Cells may be cultured inside incubators or bioreactors having an atmosphere of at least about, at most about, or about 0, 5, 10, 15, or 20% CO₂. In certain instances, satisfactory cell growth was achieved at CO₂ percentages of 5, 10, and 15%, with almost no cell growth observed when no CO₂ was provided. Typically, the growth of suspension cells require CO₂ in the culture environment and should be maintained between 1 to 20%, 5 to 15%, or any value or range there between.

Shaking speed. Optimal shaking speed was determined by the lack of foam formation and adequate suspension of the cells. Shaking speed can be from at least about, at most about, or about 5, 75, 100 to 75, 80, 100, 120 rpm. The range typically was found to be about 80-120 rpm.

Cell growth in bioreactor. In certain embodiments, a flexible bag or other type of bioreactor may be used (e.g., Wave-20 bioreactor) and seeded with suspension cells at an appropriate cell seeding density. Cells are grown inside the bioreactor. Culture condition are typically controlled and include, but are not limited to a temperature of 36.5° C., a pH at 7.20, rocking at 10 rpm. When the cell concentration reaches 2×10 cells/mL or other cell concentrations deemed appropriate, media perfusion can be initiated to allow further growth of the cells inside the bioreactor. In one example, suspension cells reached a cell concentration of approximately 2×10⁷ cells/mL at the end of the perfusion culture with good cell viability. Media perfusion may be initiated when cell concentration reaches a predetermined density (e.g., 3×10⁶ cells/mL), to allow further growth of the cells inside the bioreactor. In one example, HeLa suspension cells reached a cell concentration of more than 5×10⁷ cells/mL at the end of the perfusion culture with good cell viability. Cell growth in a bioreactor can be intensified to reach high cell concentrations by using media perfusion. The high cell concentration is expected to improve the unit productivity of adenovirus vectors.

Infection temperature. Cells may be infected at a variety of temperatures including, but not limited to at least about, at most about, or about 32° C., 33° C., 34° C., 35° C. 36° C., 37° C., 38° C. and 39° C. In certain aspects, optimal virus production is achieved at 37° C. Lower virus yield is typically seen at 32° C. and some reduction in virus production can occur at 35° C. and 39° C. In most circumstance a temperature of 37° C is used for virus production.

Multiplicity of Infection (MOI). Cells can be infected with virus at an MOI of at least about, at most about, or about 1, 5, 10, 15, 20, 25, 30, 35, 40, 45, 50, 60, 70, 80, 90. 100, 200, 300, 400, or 500 vp/cell. Virus particle concentration can be determined using a HPLC method. A relatively consistent virus yield is observed with MOIs at or above 50 vp/cell. Virus production may be reduced at MOIs lower than 50 vp/cell. Data suggest that MOIs higher than 100 did not benefit virus production and MOIs between 50-100 vp/cell appear to be the optimal range for adenovirus production in 293 suspension culture.

Infection cell density. Cells can be grown, centrifuged, and the cell pellet resuspended in fresh media at various concentrations including, but not limited to at least about, at most about, or about 5×10⁵, 1×10⁶, 1.5×10⁶, and 2×10⁶ cells/mL. The cells can then be infected with virus at a predetermined MOI. Virus particle concentration can be determined using a HPLC method. Volumetric virus yield increases with the cell density at infection. However, cell-specific virus yield decreased as the infection cell concentration increased. From an adenovirus manufacture efficiency point of view, maximize volumetric productivity is more important than obtaining high cell-specific productivity. Therefore, cells should be infected at a cell concentration that is as high as possible.

Supplementation of fresh media at virus infection. Prior to infection cells grown in a selected media can be centrifuged. Both the cell pellet and spent media supernatant may be retained. The cell pellet can be resuspended in the spent media supernatant and supplemented with different percentage of fresh media at to a desired cell concentration. Fresh media may compose at least about, at most about, or about 0, 5, 10, 15, 20, 25, 30, 35, 40, 45, 50, 5, 60, 65, 70, 75, 80, 85, 90, 95, to 100%, including all percentages there between, of the media used to resuspend the cell pellet. The virus yield data demonstrate that infection of cells in fresh media achieves a higher adenovirus production. It is possible that both nutrient limitation and metabolite product inhibition in the spent media contributed to the reduction in the adenovirus production. The data has significant implications for scale up of adenovirus production in suspension culture. One embodiment of the invention includes large scale media exchange at the time of virus infection. Mechanisms to effect media exchange include centrifugation, filtration, and fast media perfusion for a short period of time. One method is to culture cells to a high cell concentration (approximately 1×10⁷ cells/mL) using media perfusion. At the time of virus infection, dilute the concentrated culture with fresh media together with the virus for infection to achieve media exchange without using centrifugation and filtration steps.

B. Downstream Processing and Purification

Clarification filtration. Exemplary materials that may be used in a clarification procedure for crude virus harvest include, but are not limited to an Optiscale Polygard CN filter (Millipore) or similar filters and/or a Polysep II filter (Millipore) or similar filters. In certain aspects, the virus harvest is first clarified using the Polygard CN filter. A filtrate collected from the Polygard CN filter can be further filtered through a Polysep II filter. Two Polygard CN filters may be used in parallel in tandem with a Polysep II filter, the filtration rate used for the Polysep II filter can be twice that used for the Polygard CN filters. Consistent virus filtration is observed with a wide range of filtration speed and pressure. In one embodiment, the combination of two 5.0 μm Optiscale Polygard CN filters with one 0.5 μm Polysep II filter was sufficient for the clarification of crude adenovirus harvest from suspension cultures.

Concentration and Diafiltration by Tangential Flow Filtration (UFDF). Clarified virus harvest can be concentrated and diafiltered using a membrane, e.g., Millipore Pellicon II, Biomax 300KD membrane. Process parameters include membrane capacity, fold of concentration, and diafiltration efficiency. Aspects to the invention include, but is not limited to a membrane capacity of 2-6 L/1.1 ft², a concentration fold range between 5 to 20-folds. Satisfactory virus recovery was attained with a wide range of feeding flow rates. The feeding flow rates controls the transmembrane pressure of the UFDF process. Tangential flow filtration concentration and diafiltration process is robust and delivers high virus recovery and buffer exchange efficiency.

Enzyme treatment step. An endonuclease enzyme (e.g., Benzonase) treatment step may be included in the adenovirus production process to reduce the size of potential nucleic acid impurities in the final vector product. Typically, the UFDF virus material is treated with Benzonase at a concentration of 100 U/mL at room temperature for at least 16 hours. Without Benzonase treatment, significant amount of large sized DNA is seen in the UFDF material. The amount and size of DNA can be reduced by endonuclease treatment, such as Benzonase treatment. At Benzonase concentrations higher than 50 U/mL, DNA was no longer detectable on the gel after 1 hour treatment at room temperature. Endonuclease treatment may be used to reduce the amount and size of contaminating DNA.

Chromatography purification. Characterization of the chromatography purification unit operation for the Wave suspension production is still on going. Data is not yet available. However, a similar characterization study has been completed for the previous CellCube production process. Comparable results are expected from this new study.

II. Liquid Formulation

Adenoviral vectors used for human gene therapy are routinely stored at ultralow temperatures such as ≦−60° C. to maintain the long term stability of the vector. Ultralow temperature storage is expensive and not convenient for transportation and distribution. Furthermore, ultralow temperature storage is not readily available in some parts of the world and thus limits the use of adenoviral vector product in those areas.

Extensive efforts have been devoted to the development of improved formulations for adenoviral vectors. U.S. Pat. No. 6,689,600, which is incorporated herein by reference in its entirety, discloses formulations for lyophilization and liquid storage of adenoviral vector. The studies were performed at a virus concentration of approximately 1×10¹¹ vp/mL, a concentration that is 10-fold less than the current clinical concentration. Since virus aggregation is concentration dependent, the previous study did not address virus aggregation during long term storage.

Formulations disclosed by other groups all utilized sugars, such as sucrose, and divalent cations, such as Mg²⁺, in the formulation (see WO99/41416; U.S. Pat. No. 6,514,943; U.S. patent publication 20040033239, each of which is incorporated herein by reference in its entirety). On the contrary, the inventors suspect the inclusion of Mg²⁺ in a liquid formulation is detrimental to the stability of long term storage of adenovirus due to the neutralization of the negative charges present on the viral particle surfaces. The charge neutralization is expected to result in particle aggregation during long term storage. Furthermore, the presence of Mg²⁺ is expected to facilitate some of the most common protein degradation reactions, such as oxidation and deamidation. Based on the results disclosed in U.S. Pat. No. 6,689,600, the current study examines glycerol, polyethylene glycol (PEG) and Tween-80 as excipients for the formulation of adenovirus vectors using a Tris based buffer.

Embodiments of the invention are directed to development of formulations for stable storage of adenovirus products at refrigerated condition (2° C.-8° C.). Certain aspects of the invention provide for additional liquid formulations for the stability of adenovirus product at 4° C. or 25° C. storage. Previously, virus aggregation/precipitation has been identified to be one of the factors causing adenovirus instability in liquid storage. Tween-80 was found to be an effective excipient preventing the occurrence of virus precipitation in storage. In addition to virus precipitation, other factors also contributed to the virus instability in storage. One of those factors was suspected to be oxidation. The liquid formulation described herein demonstrate that oxidation is an important factor affecting adenovirus stability.

Effect of oxidation on adenovirus. Hydrogen peroxide (H₂O₂) was used as an oxidizer. Different concentrations of H₂O₂ were added to an adenovirus vector preparation at a virus concentration of 6.3×10⁶ to 6.3×10¹¹ vp/mL. After incubation at room temperature, the samples were analyzed for virus particle concentration and infectivity by a HPLC and a CPE assay, respectively. Significant reductions in virus particle concentration and infectivity were observed at H₂O₂ concentrations higher than 1%. Because of the higher sensitivity of the HPLC assay, reduction in virus particle concentration was seen even at a H₂O₂ concentration of 0.1%.

Anti-oxidation excipients. Based on the inventors' experience and literature reference alpha tocopherol; ascorbic acid; glutathione; sucrose, fructose; galactose; lactose; maltose and other sugars; ethanol; glucose; ascorbyl palmitate; ascorbyl stearate; anoxomer; butylated hydroxyanisole; butylated hydroxytoluene; citric acid; citrates; erythorbic acid and Na erythorbate; ethoxyquin; ethylenediaminetetraacetic acid; Ca disodium salt; propyl, octyl, dodecyl gallates; glycine; gum guaiac; ionox 100; (2,6-di-tert-butyl-4-hydroxymethylphenol); lecithin; polyphosphates; tartaric acid; tertiary butyl hydroquinone; trihydroxy butyrophenone; thiodipropionic acid; diauryl and distearyl esters; and arginine can be evaluated as potential anti-oxidation agents to be used in adenovirus formulations. An anti-oxidant excipient may be present as 0.01, 0.05, 0.1, 0.5, 1, 2, 3, 4, 5, 6, 7, 8, 9, 10% of an adenoviral formulation, including all values and ranges there between.

Different concentrations of ethanol can be added to an adenovirus vector preparation with a virus particle concentration of, for example, 1.2×10¹² vp/mL. H₂O₂ was added to each of the preparations to a final concentration of 1% (v/v). After 1.5 hours incubation at room temperature, the samples were analyzed by HPLC for virus particle concentration. As observed above, reduction in virus particle concentration was noticed in the presence of H₂O₂. Addition of ethanol protected the adenovirus against H₂O₂ oxidation damage. Ethanol protection was concentration dependent. Significant protection was seen at 0.5%. Ethanol may be a component of a liquid formulation in concentration of at least about, at most about, or about 0.5, 1, 2, 3, 4, 5, 6, 7, 8, 9, 10% or more and any percentage there between, of the adenoviral formulation. Overall the data indicate that ethanol is an effective anti-oxidant that could be used to stabilize adenoviral formulations.

U.S. Pat. No. 6,689,600, describes the amino acid Arginine as a possible excipient for the formulation of adenovirus. Because of the presence of an unsaturated bond in the Arginine molecule, it could be considered as a potential anti-oxidant. Similar studies to those described above for ethanol were carried out using Arginine. Different concentrations of Arginine were added to the adenovirus vector preparation. H₂O₂ was added to each of the preparations to a final concentration of 1% (v/v). After 1.5 hours incubation at room temperature, the samples were analyzed by HPLC for virus particle concentration.

Similar to that observed for ethanol, addition of Arginine protected the adenovirus against H₂O₂ oxidation damage. Protection was also concentration dependent. Significant protection was seen at 1 and 10 mM concentrations. Arginine may be a component of a liquid formulation and be present in concentration of at least about, at most about, or about 0.5, 1, 2, 3, 4, 5, 6, 7, 8, 9, 10, 11, 12, 13, 14, 15 mM or more.

Ethanol and arginine may be included in a base formulation of 20 mM Tris and/or 0.15M NaCl and/or 0.1% Tween-80 and/or 0.5% PEG, pH=8.20. Adenovirus may be formulated in those formulations at least about, at most about, or about 1×10⁵, 1×10¹⁰, 1×10¹¹, 2.5×10¹¹, 5×10¹¹, 1×10¹², 2.5×10¹², 5×10¹², 1×10¹², 2.5×10¹³, 5×10¹³, 1×10¹⁴, 2.5×10¹⁴, 5×10¹⁴, 1×10¹⁵, 2.5×10¹⁵, or 5×10¹⁵ vp/mL. The formulated virus may be stored at least about, at most about, or about 4, 5, 6, 7, 8, 9, 10, 15, 20, 25° C. and/or room temperature, which is typically 20 to 25°, for extended period of time, e.g., 5, 10, 15, 20, 25, 30 days, weeks, or months and may include 1, 2, 3, 4, 5, 6 or more years. Samples will be taken at different time points for stability assessment.

TABLE 2
Concentrations of excipients in exemplary formulation buffers

| | |
|---|---|
| Concentration of excipients | Osmolality |

| | PEG (%) | Tween-80 (%) | (mOs/L) |
|---|---|---|---|
| Formulation A | 0.5 | 0 | 310 |
| Formulation B | 0.5 | 0.1 | 307 |
| Formulation C | 0.5 | 0.5 | 298 |

Exemplary results from different formulations at different time points are shown below.

TABLE 3
Stability data for Formulation A

| | Formulation A | | | | | |
|---|---|---|---|---|---|---|
| | | | | | Par- | |
| Storage | | HPLC analysis | | | ticle | |
| temper- | Storage | R. Time | Titer | Infectivity | size | Visual |
| ature | time | (min) | (vp/mL) | (IU/mL) | (nm) | observation |
| −20° C. | 0 | 16.37 | 9.4 × 10¹¹ | 8 × 10¹⁰ | 138 | N/A |
| | 1 month | 16.12 | 7.4 × 10¹¹ | 4 × 10¹⁰ | 152 | N/A |
| | 3 month | 16.38 | 2.1 × 10¹¹ | 4 × 10¹⁰ | 186 | N/A |
| | 4 month | 16.78 | 2.0 × 10¹¹ | 4 × 10¹⁰ | 280 | N/A |
| 2-8° C. | 0 | 16.37 | 9.4 × 10¹¹ | 8 × 10¹⁰ | 138 | No |
| | | | | | | precipitation |
| | 1 month | 16.27 | 9.6 × 10¹¹ | 8 × 10¹⁰ | 139 | Precipitation |
| | 3 month | 16.55 | 5.1 × 10¹¹ | 8 × 10¹⁰ | 153 | Precipitation |
| | 4 month | 16.88 | 2.8 × 10¹¹ | 2 × 10¹⁰ | 205 | Precipitation |
| 25° C. | 0 | 16.37 | 9.4 × 10¹¹ | 8 × 10¹⁰ | 138 | No |
| | | | | | | precipitation |
| | 1 week | 16.73 | 9.6 × 10¹¹ | 4 × 10¹⁰ | 140 | No |
| | | | | | | Precipitation |
| | 1 month | 16.67 | 8.0 × 10¹¹ | 2 × 10¹⁰ | 163 | Precipitation |
| | 3 month | 16.86 | 4.2 × 10¹¹ | 2 × 10¹⁰ | 170 | Precipitation |
| | 4 month | 17.10 | 4.2 × 10¹¹ | 1 × 10⁹ | 173 | Precipitation |

TABLE 4
Stability data For Formulation B

| | Formulation B | | | | | |
|---|---|---|---|---|---|---|
| | | HPLC | | | Par- | |
| Storage | | R. | | | ticle | |
| temper- | Storage | time | Titer | Infectivity | size | Visual |
| ature | time | (min) | (vp/mL) | (IU/mL) | (nm) | observation |
| −20° C. | 0 | 16.30 | 9.8 × 10¹¹ | 8 × 10¹⁰ | 132 | N/A |
| | 1 month | 16.58 | 8.7 × 10¹¹ | 8 × 10¹⁰ | 148 | N/A |
| | 3 month | 16.57 | 3.2 × 10¹¹ | 8 × 10¹⁰ | 135 | N/A |
| | 4 month | No | No peak | 1 × 10⁸ | 150 | N/A |
| | | peak | | | | |
| 2-8° C. | 0 | 16.30 | 9.8 × 10¹¹ | 8 × 10¹⁰ | 132 | No |
| | | | | | | precipitation |
| | 1 month | 16.32 | 1.0 × 10¹² | 8 × 10¹⁰ | 127 | No |
| | | | | | | precipitation |
| | 3 month | 16.72 | 3.3 × 10¹¹ | 4 × 10¹⁰ | 129 | No |
| | | | | | | precipitation |
| | 4 month | No | No peak | 1 × 10¹⁰ | 164 | No |
| | | peak | | | | precipitation |
| 25° C. | 0 | 16.30 | 9.8 × 10¹¹ | 8 × 10¹⁰ | 132 | No |
| | | | | | | precipitation |
| | 1 week | 16.52 | 1.1 × 10¹² | 8 × 10¹⁰ | 114 | No |
| | | | | | | precipitation |
| | 1 month | 16.58 | 9.0 × 10¹¹ | 8 × 10¹⁰ | 114 | No |
| | | | | | | precipitation |
| | 3 month | 17.85 | 5.9 × 10¹¹ | 8 × 10¹⁰ | 117 | No |
| | | | | | | precipitation |
| | 4 month | 17.02 | 5.1 × 10¹¹ | 1 × 10⁹ | 117 | No |
| | | | | | | precipitation |

TABLE 5
Stability data For Formulation C

| | Formulation C | | | | | |
|---|---|---|---|---|---|---|
| | | HPLC | | | Par- | |
| Storage | | R. | | | ticle | |
| temper- | Storage | time | Titer | Infectivity | size | Visual |
| ature | time | (min) | (vp/mL) | (IU/mL) | (nm) | observation |
| −20° C. | 0 | 16.44 | 1.1 × 10¹² | 8 × 10¹⁰ | 125 | N/A |
| | 1 month | 16.42 | 9.8 × 10¹¹ | 8 × 10¹⁰ | 122 | N/A |
| | 3 month | 16.76 | 2.6 × 10¹¹ | 8 × 10¹⁰ | 129 | N/A |
| | 4 month | 16.97 | 2.6 × 10¹¹ | 2 × 10¹⁰ | 125 | N/A |
| 2-8° C. | 0 | 16.44 | 1.1 × 10¹² | 8 × 10¹⁰ | 125 | No |
| | | | | | | precipitation |
| | 1 month | 16.27 | 9.5 × 0¹¹ | 8 × 10¹⁰ | 111 | No |
| | | | | | | precipitation |
| | 3 month | 16.77 | 2.2 × 10¹¹ | 4 × 10¹⁰ | 120 | No |
| | | | | | | precipitation |
| | 4 month | No | No peak | 1 × 10⁹ | 170 | No |
| | | peak | | | | precipitation |
| 25° C. | 0 | 16.44 | 1.1 × 10¹² | 8 × 10¹⁰ | 125 | No |
| | | | | | | precipitation |
| | 1 week | 16.44 | 1.1 × 10¹² | 8 × 10¹⁰ | 107 | No |
| | | | | | | precipitation |
| | 1 month | 16.52 | 9.0 × 10¹¹ | 8 × 10¹⁰ | 112 | No |
| | | | | | | precipitation |
| | 3 month | 17.30 | 4.9 × 10¹¹ | 8 × 10¹⁰ | 113 | No |
| | | | | | | precipitation |
| | 4 month | 17.09 | 4.5 × 10¹¹ | 1 × 10⁹ | 110 | No |
| | | | | | | precipitation |

In the formulation that does not contain Tween-80 (Formulation A), increase in particle size was observed after I month storage. The increase in particle size is believed to have caused the precipitation seen in the vials stored at 2-8° C. and 25° C. After 4 month storage at 25° C., virus infectivity decreased approximately 2 logs. Total virus particle concentration analyzed by HPLC also decreased. For storage temperatures of 2-8° C. and −20° C., similar loss of virus infectivity and virus particle concentration were observed.

For the formulations that contain Tween-80 (Formulation B and C), virus remained stable after 1 month storage at 25° C. No increases in particle size and virus precipitation were observed. The result suggests that the presence of Tween-80 in the formulation prevented virus precipitation in non-frozen, liquid storage and extended the stability of the adenovirus product. Similar stability data were seen at −20° C. and 2-8° C. storage.

Loss of virus stability was observed at 3 and 4 month storage time points for both Formulation B and C under all three storage temperatures. It appears that most of the decrease in virus infectivity occurred between 3 and 4 months of storage. A decrease in virus particle concentration was also noticed by HPLC analysis. The decrease in virus stability is not caused by virus aggregation/precipitation as no appreciable change in virus particle size was observed and no visible precipitation was seen in the container. Possible mechanisms for the loss of virus stability are oxidation, deglycosylation, and deamidation of virus proteins. The fact that PEG and Tween-80, which are prone to contain trace amount of peroxide, are included in the formulations makes oxidation a likely mechanism for the loss of virus infectivity.

For 25° C. storage condition, an increase in the HPLC retention time was seen as the virus titer decreased. It appears that both the infectivity and the HPLC assays are able to detect changes in virus stability during storage, thus are stability indicating assays. On the other hand, results from the particle size assay do not correlate with the stability of the virus and is not a stability indicating assay.

These formulations indicate that inclusion of Tween-80 in the liquid formulation helped to prevent virus aggregation/precipitation during storage at 2-8° C. and 25° C. In formulations containing Tween-80, virus maintained stability at 25° C. for up to and more than one month at a virus concentration of 1×10¹² vp/mL.

III. Adenovirus

Adenoviruses comprise linear double stranded DNA, with a genome ranging from 30 to 36 kb in size (Reddy et al., 1998; Morrison et al., 1997; Chillon et al., 1999). There are over 50 serotypes of human adenovirus, and over 80 related forms which are divided into six families based on immunological, molecular, and functional criteria (Wadell et al., 1980). Adenovirus is a medium-sized icosahedral virus containing a double-stranded, linear DNA genome, which, for adenovirus type 5, is 35,935 base pairs (Chroboczek et al., 1992). Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a sequence (Zheng, et al., 1999; Robbins et al. , 1998; Graham and Prevec, 1995).

In certain embodiments of the present invention, an adenovirus may be a replication-deficient or replication competent adenovirus. For example, the adenovirus may be a replication-deficient adenovirus lacking at least a portion of the E1 region. In certain embodiments, the adenovirus may be lacking at least a portion of the E1A and/or E1B region. In other embodiments, the adenovirus is a recombinant adenovirus (discussed further below).

A. Host Cells

Various embodiments of the present invention involve methods for producing an adenovirus. A “host cell” is defined as a cell that is capable of supporting replication of adenovirus. Any cell type for use as a host cell is contemplated by the present invention, as long as the cell is capable of supporting replication of adenovirus. For example, the host cells may be HEK293, PER.C6, 911, or IT293SF cells. One of skill in the art would be familiar with the wide range of host cells that are available for use in methods for producing an adenovirus.

In certain embodiments, the generation and propagation of the adenoviral vectors depend on a unique helper cell line, designated 293, which was transformed from human embryonic kidney cells by Adenovirus serotype 5 (Ad5) DNA fragments and constitutively expresses E1 proteins (Graham et al., 1977). Since the E3 region is dispensable from the Ad genome (Jones and Shenk, 1978), the current Ad vectors, with the help of 293 cells, carry foreign DNA in either the E1, the E3 or both regions (Graham and Prevec, 1991; Bett et al., 1994).

The host cells used in the various embodiments of the present invention may be derived, for example, from mammalian cells such as human embryonic kidney cells or primate cells. Other cell types might include, but are not limited to Vero cells, CHO cells or any eukaryotic cells for which tissue culture techniques are established as long as the cells are adenovirus permissive. The term “adenovirus permissive” means that the adenovirus or adenoviral vector is able to complete the entire intracellular virus life cycle within the cellular environment.

The host cell may be derived from an existing cell line, e.g., from a 293 cell line, or developed de novo. Such host cells express the adenoviral genes necessary to complement in trans deletions in an adenoviral genome or which supports replication of an otherwise defective adenoviral vector, such as the E1, E2, E4, E5 and late functions. A particular portion of the adenovirus genome, the E1 region, has already been used to generate complementing cell lines. Whether integrated or episomal, portions of the adenovirus genome lacking a viral origin of replication, when introduced into a cell line, will not replicate even when the cell is superinfected with wild-type adenovirus. In addition, because the transcription of the major late unit is after viral DNA replication, the late functions of adenovirus cannot be expressed sufficiently from a cell line. Thus, the E2 regions, which overlap with late functions (L1-5), will be provided by helper viruses and not by the cell line. Typically, a cell line according to the present invention will express E1 and/or E4.

Recombinant host cells, which are host cells that express part of the adenoviral genome, are also contemplated for use as host cells in the present invention. As used herein, the term “recombinant” cell is intended to refer to a cell into which a gene, such as a gene from the adenoviral genome or from another cell, has been introduced. Therefore, recombinant cells are distinguishable from naturally-occurring cells which do not contain a recombinantly-introduced gene. Recombinant cells are thus cells having a gene or genes introduced through “the hand of man.”

Recombinant host cells lines are capable of supporting replication of adenovirus recombinant vectors and helper viruses having defects in certain adenoviral genes, i.e., are “permissive” for growth of these viruses and vectors. The recombinant cell also is referred to as a helper cell because of the ability to complement defects in, and support replication of, replication-incompetent adenoviral vectors.

Examples of other useful mammalian cell lines that may be used with a replication competent virus or converted into complementing host cells for use with replication deficient virus are Vero and HeLa cells and cell lines of Chinese hamster ovary, W138, BHK, COS-7, HepG2, 3T3, RIN and MDCK cells.

Two methodologies have been used to adapt 293 cells into suspension cultures. Graham adapted 293A cells into suspension culture (293N3S cells) by 3 serial passages in nude mice (Graham, 1987). The suspension 293N3S cells were found to be capable of supporting E1-adenoviral vectors. However, Gamier et al. (1994) observed that the 293N35 cells had a relatively long initial lag phase in suspension, a low growth rate, and a strong tendency to clump.

The second method that has been used is a gradual adaptation of 293A cells into suspension growth (Cold Spring Harbor Laboratories, 293S cells). Gamier et al. (1994) reported the use of 293S cells for production of recombinant proteins from adenoviral vectors. The authors found that 293S cells were much less clumpy in calcium-free media and a fresh medium exchange at the time of virus infection could significantly increase the protein production. It was found that glucose was the limiting factor in culture without medium exchange.

1. Growth in Selection Media

In certain embodiments, it may be useful to employ selection systems that preclude growth of undesirable cells. This may be accomplished by virtue of permanently transforming a cell line with a selectable marker or by transducing or infecting a cell line with a viral vector that encodes a selectable marker. In either situation, culture of the transformed/transduced cell with an appropriate drug or selective compound will result in the enhancement, in the cell population, of those cells carrying the marker.

Examples of markers include, but are not limited to, HSV thymidine kinase, hypoxanthine-guanine phosphoribosyltransferase, and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; and hygro, that confers resistance to hygromycin.

2. Growth in Serum Weaning

Serum weaning adaptation of anchorage-dependent cells into serum-free suspension cultures have been used for the production of recombinant proteins (Berg, 1993) and viral vaccines (Perrin, 1995). There have been few reports on the adaptation of 293A cells into serum-free suspension cultures until recently. Gilbert reported the adaptation of 293A cells into serum-free suspension cultures for adenovirus and recombinant protein production (Gilbert, 1996). Similar adaptation method had been used for the adaptation of A549 cells into serum-free suspension culture for adenovirus production (Morris et al., 1996). Cell-specific virus yields in the adapted suspension cells, however, are about 5-10-fold lower than those achieved in the parental attached cells.

Using the similar serum weaning procedure, the inventors have successfully adapted the 293A cells into serum-free suspension culture (293SF cells). In this procedure, the 293 cells were adapted to a commercially available 293 media by sequentially lowering down the FBS concentration in T-flasks. Briefly, the initial serum concentration in the media was approximately 10% FBS DMEM media in T-75 flask and the cells were adapted to serum-free IS 293 media in T-flasks by lowering down the FBS concentration in the media sequentially. After 6 passages in T-75 flasks the FBS % was estimated to be about 0.019% and the 293 cells. The cells were subcultured two more times in the T flasks before they were transferred to spinner flasks. The results described herein below show that cells grow satisfactorily in the serum-free medium (IS293 medium, Irvine Scientific, Santa Ana, Calif.). Average doubling time of the cells was 20-35 hours achieving stationary cell concentrations in the order of 3-5×10⁶ cells/ml without medium exchange.

3. Adaptation of Cells for Suspension Culture

Two methodologies have been used to adapt 293 cells into suspension cultures. Graham adapted 293A cells into suspension culture (293N3S cells) by 3 serial passages in nude mice (Graham, 1987). The suspension 293N3S cells were found to be capable of supporting El-adenoviral vectors. However, Gamier et al. (1994) observed that the 293N35 cells had a relatively long initial lag phase in suspension, a low growth rate, and a strong tendency to clump.

The second method that has been used is a gradual adaptation of 293A cells into suspension growth (Cold Spring Harbor Laboratories, 293S cells). Gamier et al. (1994) reported the use of 293S cells for production of recombinant proteins from adenoviral vectors. The authors found that 293S cells were much less clumpy in calcium-free media and a fresh medium exchange at the time of virus infection could significantly increase the protein production. It was found that glucose was the limiting factor in culture without medium exchange.

In the present invention, the 293 cells adapted for growth in serum-free conditions were adapted into a suspension culture. The cells were transferred in a serum-free 250 mL spinner suspension culture (100 mL working volume) for the suspension culture at an initial cell density of between about 1.18×10⁵ vc/mL and about 5.22×10⁵ vc/mL. The media may be supplemented with heparin to prevent aggregation of cells. This cell culture systems allows for some increase of cell density whilst cell viability is maintained. Once these cells are growing in culture, they cells are subcultured in the spinner flasks approximately 7 more passages. It may be noted that the doubling time of the cells is progressively reduced until at the end of the successive passages the doubling time is about 1.3 day, i.e., comparable to 1.2 day of the cells in 10% FBS media in the attached cell culture. In the serum-free IS 293 media supplemented with heparin almost all the cells existed as individual cells not forming aggregates of cells in the suspension culture.

B. Cell Culture Systems

1. High Producer Cell Density and High Virus Yield.

Microcarrier cell culture in stirred tank bioreactor provides very high volume-specific culture surface area and has been used for the production of viral vaccines (Griffiths, 1986). Furthermore, stirred tank bioreactors have industrially been proven to be scaleable. One example is the multiplate CellCube™ cell culture system. The ability to produce infectious viral vectors is increasingly important to the pharmaceutical industry, especially in the context of gene therapy. Over the last decade, advances in biotechnology have led to the production of a number of important viral vectors that have potential uses as therapies, vaccines and protein production machines.

Frequently, factors which affect the downstream (in this case, beyond the cell lysis) side of manufacturing scale-up were not considered before selecting the cell line as the host for the expression system. Also, development of bioreactor systems capable of sustaining very high density cultures for prolonged periods of time have not lived up to the increasing demand for increased production at lower costs.

The present invention will take advantage of the recently available bioreactor technology. Growing cells according to the present invention in a bioreactor allows for large scale production of fully biologically-active cells capable of being infected by the adenoviral vectors of the present invention. By operating the system at a low perfusion rate and applying a different scheme for purification of the infecting particles, the invention provides a purification strategy that is easily scaleable to produce large quantities of highly purified product.

PCT publication WO 98/00524, U.S. Pat. No. 6,194,191, U.S. Patent publication 20020182723, and U.S. Provisional Patent Application No. 60/406,591 (filed Aug. 28, 2002), which have described viral production methods, are specifically incorporated by reference for their description of techniques for culturing, production, and purification of recombinant viral particles.

Certain embodiments of the present invention pertain to methods for producing an adenovirus that require the use of a bioreactor. As used herein, a “bioreactor” refers to any apparatus that can be used for the purpose of culturing cells. Growing cells according to the present invention in a bioreactor allows for large scale production of fully biologically-active cells capable of being infected by the adenoviral vectors of the present invention.

Bioreactors have been widely used for the production of biological products from both suspension and anchorage dependent animal cell cultures. The most widely used producer cells for adenoviral vector production are anchorage dependent human embryonic kidney cells (293 cells). Bioreactors to be developed for adenoviral vector production should have the characteristic of high volume-specific culture surface area in order to achieve manufactured by Coming-Costar also offers a very high volume-specific culture surface area. Cells grow on both sides of the culture plates hermetically sealed together in the shape of a compact cube. Unlike stirred tank bioreactors, the CellCube™ culture unit is disposable. This is very desirable at the early stage production of clinical product because of the reduced capital expenditure, quality control and quality assurance costs associated with disposable systems. In consideration of the advantages offered by the different systems, both the stirred tank bioreactor and the CellCube™ system were evaluated for the production of adenovirus.

Certain embodiments of the present invention require the use of a Wave Bioreactor®, particularly for use in methods for generating adenovirus in serum-free suspension cultures. The Wave Bioreactor® is a pre-sterilized disposable bioreactor system that can be easily retrofitted with a variety of clean room configurations without requiring expensive CIP and SIP process utilities. The Wave Bioreactor® can be a Wave Biotech® model 20/50EH. The bioreactor can hold any volume of media, but in a certain embodiment the bioreactor is a 10 L (5 L working volume) bioreactor. In certain embodiments, the bioreactor can be adjusted to rock at a particular speed and angle. In certain other embodiments, the bioreactor may include a device for monitoring dissolved oxygen tension, such as a disposable dissolved oxygen tension (DOT) probe. The bioreactor may also include a device for monitoring temperature in the media. Other embodiments include a device for measuring and adjusting culture pH, such as a gas mixer which can adjust CO₂ gas percentage delivered to the media. The bioreactor may or may not be a disposable bioreactor. According to an aspect of the invention, the Wave Bioreactor® is used with serum-free media and the initial lactate concentration of the medium is made as low as possible because high lactate concentration inhibits virus production. Further, an adequate glucose concentration should be maintained as glucose limitation can also inhibit virus production. As used herein, “media” and “medium” refers to any substance which can facilitate growth of cells. According to one aspect of the present invention, the host cells are grown in media that is serum-free media. In other embodiments of the present invention, the host cells are grown in media that is protein-free media. One example of a protein-free media is CD293. Another example of media that can support host cell growth in a particular embodiment of the invention is DMEM+2% FBS. On of skill in the art would understand that various components and agents can be added to the media to facilitate and control cell growth. For example, the glucose concentration of the media can be maintained at a certain level. In one embodiment of the present methods for producing adenovirus, the glucose concentration is maintained between about 0.5 and about 3.0 gm glucose/liter.

2. Anchorage-Dependent Versus Non-Anchorage-Dependent Cultures

In some embodiments of the present invention, the methods for producing an adenovirus require growing host cells in anchorage-dependent cultures, whereas other embodiments pertain to methods for producing an adenovirus in non-anchorage-dependent cultures. Animal and human cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. Large scale suspension culture based on microbial (bacterial and yeast) fermentation technology has clear advantages for the manufacturing of mammalian cell products. The processes are relatively simple to operate and straightforward to scale up. Homogeneous conditions can be provided in the reactor which allows for precise monitoring and control of temperature, dissolved oxygen, and pH, and ensure that representative samples of the culture can be taken.

However, suspension cultured cells cannot always be used in the production of biologicals. Suspension cultures are still considered to have tumorigenic potential and thus their use as substrates for production put limits on the use of the resulting products in human and veterinary applications (Petricciani, 1985; Larsson, 1987). Viruses propagated in suspension cultures as opposed to anchorage-dependent cultures can sometimes cause rapid changes in viral markers, leading to reduced immunogenicity (Bahnemann, 1980). Finally, sometimes even recombinant cell lines can secrete considerably higher amounts of products when propagated as anchorage-dependent cultures as compared with the same cell line in suspension (Nilsson and Mosbach, 1987). For these reasons, different types of anchorage-dependent cells are used extensively in the production of different biological products.

3. Reactors and Processes for Suspension

The bioreactors utilized in the context of selected embodiments of the present invention may be stirred tank bioreactors. Large scale suspension culture of mammalian cultures in stirred tanks have been described. The instrumentation and controls for bioreactors adapted, along with the design of the fermentors, from related microbial applications. However, acknowledging the increased demand for contamination control in the slower growing mammalian cultures, improved aseptic designs were quickly implemented, improving dependability of these reactors. Instrumentation and controls are basically the same as found in other fermentors and include agitation, temperature, dissolved oxygen, and pH controls. More advanced probes and autoanalyzers for on-line and off-line measurements of turbidity (a function of particles present), capacitance (a function of viable cells present), glucose/lactate, carbonate/bicarbonate and carbon dioxide are available. In one embodiment of the present invention, the autoanalyzer is a YSI-2700 SELECT™ analyzer.

Two suspension culture reactor designs are most widely used in the industry due to their simplicity and robustness of operation--the stirred reactor and the airlift reactor. The stirred reactor design has successfully been used on a scale of 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcorner section of the reactor. The main advantage of this design is the simplicity and lack of need for mechanical mixing. Typically, the height-to-diameter ratio is 10:1. The airlift reactor scales up relatively easily, has good mass transfer of gasses and generates relatively low shear forces.

Most large-scale suspension cultures are operated as batch or fed-batch processes because they are the most straightforward to operate and scale up. However, continuous processes based on chemostat or perfusion principles are available.

A batch process is a closed system in which a typical growth profile is seen. A lag phase is followed by exponential, stationary and decline phases. In such a system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. Such a fed-batch process is still a closed system because cells, products and waste products are not removed.

In what is still a closed system, perfusion of fresh medium through the culture can be achieved by retaining the cells with a variety of devices (e.g., fine mesh spin filter, hollow fiber or flat plate membrane filters, settling tubes). Spin filter cultures can produce cell densities of approximately 5×10⁷ cells/ml. A true open system and the simplest perfusion process is the chemostat in which there is an inflow of medium and an outflow of cells and products. Culture medium is fed to the reactor at a predetermined and constant rate which maintains the dilution rate of the culture at a value less than the maximum specific growth rate of the cells (to prevent washout of the cell mass from the reactor). Culture fluid containing cells and cell products and byproducts is removed at the same rate.

In certain embodiments of the present methods for producing adenovirus, the bioreactor system is set up to include a system to allow for media exchange. For example, filters may be incorporated into the bioreactor system to allow for separation of cells from spent media to facilitate media exchange. In some embodiments of the present methods for producing adenovirus, media exchange and perfusion is conducted beginning on a certain day of cell growth. For example, media exchange and perfusion can begin on day 3 of cell growth. The filter may be external to the bioreactor, or internal to the bioreactor.

In one embodiment of the present invention, the filter is a floating flat filter that is internal to the bioreactor. The filter provides for separation between the cells and spent medium. In certain embodiments, the spent culture media is withdrawn through the floating filer. Recirculation of the media may or may not be required in the various embodiments of the present invention. In one embodiment, wave action is used to minimize clogging of the filter during media perfusion. The culture volume may be maintained by a load cell used to trigger fresh medium addition. One of skill in the art would be familiar with the various types of filters that can be used for perfusion of media, and the various methods that can be employed for attaching the filter to the bioreactor and incorporating it into the cell growth process.

4. Non-Perfused Attachment Systems

Traditionally, anchorage-dependent cell cultures are propagated on the bottom of small glass or plastic vessels. The restricted surface-to-volume ratio offered by classical and traditional techniques, suitable for the laboratory scale, has created a bottleneck in the production of cells and cell products on a large scale. In an attempt to provide systems that offer large accessible surfaces for cell growth in small culture volume, a number of techniques have been proposed: the roller bottle system, the stack plate's propagator, the spiral film bottles, the hollow fiber system, the packed bed, the plate exchanger system, and the membrane tubing reel. Since these systems are non-homogeneous in their nature, and are sometimes based on multiple processes, they suffer from the following shortcomings--limited potential for scale-up, difficulties in taking cell samples, limited potential for measuring and controlling key process parameters and difficulty in maintaining homogeneous environmental conditions throughout the culture.

Despite these drawbacks, a commonly used process for large scale anchorage-dependent cell production is the roller bottle. Being little more than a large, differently shaped T-flask, simplicity of the system makes it very dependable and, hence, attractive. Fully automated robots are available that can handle thousands of roller bottles per day, thus eliminating the risk of contamination and inconsistency associated with the otherwise required intense human handling. With frequent media changes, roller bottle cultures can achieve cell densities of close to 0.5×10⁶ cells/cm² (corresponding to approximately 10⁹ cells/bottle or almost 10⁷ cells/ml of culture media).

5. Cultures on Microcarriers

In an effort to overcome the shortcomings of the traditional anchorage-dependent culture processes, van Wezel (1967) developed the concept of the microcarrier culturing systems. In this system, cells are propagated on the surface of small solid particles suspended in the growth medium by slow agitation. Cells attach to the microcarriers and grow gradually to confluency on the microcarrier surface. In fact, this large scale culture system upgrades the attachment dependent culture from a single disc process to a unit process in which both monolayer and suspension culture have been brought together. Thus, combining the necessary surface for a cell to grow with the advantages of the homogeneous suspension culture increases production.

The advantages of microcarrier cultures over most other anchorage-dependent, large-scale cultivation methods are several fold. First, microcarrier cultures offer a high surface-to-volume ratio (variable by changing the carrier concentration) which leads to high cell density yields and a potential for obtaining highly concentrated cell products. Cell yields are up to 1-2×10⁷ cells/ml when cultures are propagated in a perfused reactor mode. Second, cells can be propagated in one unit process vessels instead of using many small low-productivity vessels (i.e., flasks or dishes). This results in far better nutrient utilization and a considerable saving of culture medium. Moreover, propagation in a single reactor leads to reduction in need for facility space and in the number of handling steps required per cell, thus reducing labor cost and risk of contamination. Third, the well-mixed and homogeneous microcarrier suspension culture makes it possible to monitor and control environmental conditions (e.g., pH, p02, and concentration of medium components), thus leading to more reproducible cell propagation and product recovery. Fourth, it is possible to take a representative sample for microscopic observation, chemical testing, or enumeration. Fifth, since microcarriers settle out of suspension quickly, use of a fed-batch process or harvesting of cells can be done relatively easily. Sixth, the mode of the anchorage-dependent culture propagation on the microcarriers makes it possible to use this system for other cellular manipulations, such as cell transfer without the use of proteolytic enzymes, cocultivation of cells, transplantation into animals, and perfusion of the culture using decanters, columns, fluidized beds, or hollow fibers for microcarrier retainment. Seventh, microcarrier cultures are relatively easily scaled up using conventional equipment used for cultivation of microbial and animal cells in suspension.

6. Microencapsulation of Mammalian Cells

One method which has shown to be particularly useful for culturing mammalian cells is microencapsulation. The mammalian cells are retained inside a semipermeable hydrogel membrane. A porous membrane is formed around the cells permitting the exchange of nutrients, gases, and metabolic products with the bulk medium surrounding the capsule. Several methods have been developed that are gentle, rapid and non-toxic and where the resulting membrane is sufficiently porous and strong to sustain the growing cell mass throughout the term of the culture. These methods are all based on soluble alginate gelled by droplet contact with a calcium-containing solution. U.S. Pat. No. 4,352,883, incorporated herein by reference, describes cells concentrated in an approximately 1% solution of sodium alginate which are forced through a small orifice, forming droplets, and breaking free into an approximately 1% calcium chloride solution. The droplets are then cast in a layer of polyamino acid that ionically bonds to the surface alginate. Finally the alginate is reliquefied by treating the droplet in a chelating agent to remove the calcium ions. Other methods use cells in a calcium solution to be dropped into a alginate solution, thus creating a hollow alginate sphere. A similar approach involves cells in a chitosan solution dropped into alginate, also creating hollow spheres.

Microencapsulated cells are easily propagated in stirred tank reactors and, with beads sizes in the range of 150-1500 mm in diameter, are easily retained in a perfused reactor using a fine-meshed screen. The ratio of capsule volume to total media volume can be maintained from as dense as 1:2 to 1:10. With intracapsular cell densities of up to 10⁸, the effective cell density in the culture is 1-5×10⁷.

The advantages of microencapsulation over other processes include the protection from the deleterious effects of shear stresses which occur from sparging and agitation, the ability to easily retain beads for the purpose of using perfused systems, scale up is relatively straightforward and the ability to use the beads for implantation.

The current invention includes cells which are anchorage-dependent in nature. 293 cells, for example, are anchorage-dependent, and when grown in suspension, the cells will attach to each other and grow in clumps, eventually suffocating cells in the inner core of each clump as they reach a size that leaves the core cells unsustainable by the culture conditions. Therefore, an efficient means of large-scale culture of anchorage-dependent cells is needed in order to effectively employ these cells to generate large quantities of adenovirus.

7. Perfused Attachment Systems

Certain embodiments of the present invention involve methods for producing an adenovirus that involve use of perfused attachment systems. Perfusion refers to continuous flow at a steady rate, through or over a population of cells (of a physiological nutrient solution). It implies the retention of the cells within the culture unit as opposed to continuous-flow culture which washes the cells out with the withdrawn media (e.g., chemostat). The idea of perfusion has been known since the beginning of the century, and has been applied to keep small pieces of tissue viable for extended microscopic observation. The technique was initiated to mimic the cells milieu in vivo where cells are continuously supplied with blood, lymph, or other body fluids. Without perfusion, cells in culture go through alternating phases of being fed and starved, thus limiting full expression of their growth and metabolic potential.

The current use of perfused culture is in response to the challenge of growing cells at high densities (i.e., 0.1-5×10⁸ cells/ml). In order to increase densities beyond 2-4×10⁶ cells/ml, the medium has to be constantly replaced with a fresh supply in order to make up for nutritional deficiencies and to remove toxic products. Perfusion allows for a far better control of the culture environment (pH, pO₂, nutrient levels, etc.) and is a means of significantly increasing the utilization of the surface area within a culture for cell attachment.

The development of a perfused packed-bed reactor using a bed matrix of a non-woven fabric has provided a means for maintaining a perfusion culture at densities exceeding 10⁸ cells/ml of the bed volume (CelliGen™, New Brunswick Scientific, Edison, N.J.; Wang et al., 1992; Wang et al., 1993; Wang et al., 1994). Briefly described, this reactor comprises an improved reactor for culturing of both anchorage- and non-anchorage-dependent cells. The reactor is designed as a packed bed with a means to provide internal recirculation. A fiber matrix carrier can be placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 mm to 100 mm, providing for a high internal volume with pore volumes corresponding to 1 to 20 times the volumes of individual cells.

In comparison to other culturing systems, this approach offers several significant advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium which facilitates subsequent purification steps. Also, the unique design of this reactor system offers an easier way to scale up the reactor. Currently, sizes up to 30 liter are available. One hundred liter and 300 liter versions are in development and theoretical calculations support up to a 1000 liter reactor. This technology is explained in detail in WO 94/17178, incorporated by reference in its entirety.

The CellCube™ (Corning-Costar) module provides a large styrenic surface area for the immobilization and growth of substrate attached cells. It is an integrally encapsulated sterile single-use device that has a series of parallel culture plate joined to create thin sealed laminar flow spaces between adjacent plates.

The CellCube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrients composition at a variety of different perfusion rates to determine the most economical and productive operating parameters.

Cells within the system reach a higher density of solution (cells/ml) than in traditional culture systems. Many typically used basal media are designed to support 1-2×10⁶ cells/ml/day. A typical CellCube™ run with an 85,000 cm² surface, contains approximately 6 L media within the module. The cell density often exceeds 107 cells/mL in the culture vessel. At confluence, 2-4 reactor volumes of media are required per day.

The timing and parameters of the production phase of cultures depends on the type and use of a particular cell line. Many cultures require a different media for production than is required for the growth phase of the culture. The transition from one phase to the other will likely require multiple washing steps in traditional cultures. However, the CellCube™ system employs a perfusion system. One of the benefits of such a system is the ability to provide a gentle transition between various operating phases. The perfusion system negates the need for traditional wash steps that seek to remove serum components in a growth medium.

8. Serum-Free Suspension Culture

In particular embodiments, adenoviral vectors for gene therapy are produced from anchorage-dependent culture of 293 cells (293A cells) as described above. Scale-up of adenoviral vector production is constrained by the anchorage-dependency of 293A cells. To facilitate scale-up and meet future demand for adenoviral vectors, significant efforts have been devoted to the development of alternative production processes that are amenable to scale-up. Methods include growing 293A cells in microcarrier cultures and adaptation of 293A producer cells into suspension cultures.

Microcarrier culture techniques have been described above. This technique relies on the attachment of producer cells onto the surfaces of microcarriers which are suspended in culture media by mechanical agitation. The requirement of cell attachment may present some limitations to the scalability of microcarrier cultures.

In certain embodiments of the present invention, the media used in the methods for producing an adenovirus is a serum-free media. In other embodiments of the present invention, the media is a protein-free media. As previously discussed, certain embodiments of the present invention involve use of bioreactors. The bioreactors may be adapted for serum-free suspension culture of cells. Filtration of media with media exchange may or may not be included in the system.

C. Viral Infection

The present invention includes methods of producing an adenovirus by infecting a host cells with an adenovirus. Typically, the virus will simply be exposed to the appropriate host cell under physiologic conditions, permitting uptake of the virus. One of skill in the art would be familiar with the wide range of techniques available for initiating virus infection.

The present invention employs, in one example, adenoviral infection of cells in order to generate therapeutically significant vectors. Typically, the virus will simply be exposed to the appropriate host cell under physiologic conditions, permitting uptake of the virus. Though adenovirus is exemplified, the present methods may be advantageously employed with other viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants. Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

2. Retrovirus

Although adenoviral infection of cells for the generation of therapeutically significant vectors is an embodiment of the present invention, it is contemplated that the present invention may employ retroviral infection of cells for the purposes of generating such vectors. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Y, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Y components is constructed (Mann et al., 1983). When a recombinant plasmid containing a human cDNA, together with the retroviral LTR and Y sequences is introduced into this cell line (by calcium phosphate precipitation for example), the Y sequence allows the RNA transcript of the recombinant plasmid to be packaged into viral particles, which are then secreted into the culture media (Nicolas and Rubenstein, 1988; Temin, 1986; Mann et al., 1983).

3. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), herpes viruses and lentivirus may be employed. These viruses offer several features for use in gene transfer into various mammalian cells.

IV. Methods of Gene Transfer

In order to create the helper cell lines of the present invention, and to create recombinant adenovirus vectors for use therewith, various genetic (i.e., DNA) constructs must be delivered to a cell. One way to achieve this is via viral transductions using infectious viral particles, for example, by transformation with an adenovirus vector of the present invention. Alternatively, retroviral or bovine papilloma virus may be employed, both of which permit permanent transformation of a host cell with a gene(s) of interest. In other situations, the nucleic acid to be transferred is not infectious, i.e., contained in an infectious virus particle. This genetic material must rely on non-viral methods for transfer.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter -et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988)

Once the construct has been delivered into the cell the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle.

In one embodiment of the invention, the expression construct may simply consist of naked recombinant DNA or plasmids. Transfer of the construct may be performed by any of the methods mentioned above which physically or chemically permeabilize the cell membrane. This is particularity applicable for transfer in vitro, however, it may be applied for in vivo use as well.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

An expression construct may be entrapped in a liposome. Liposomes are vesicular structures characterized by a phospholipid bilayer membrane and an inner aqueous medium. Multilamellar liposomes have multiple lipid layers separated by aqueous medium. Liposome-mediated nucleic acid delivery and expression of foreign DNA in vitro has been very successful. Using the μ-lactamase gene, Wong et al. (1980) demonstrated the feasibility of liposome-mediated delivery and expression of foreign DNA in cultured chick embryo, HeLa, and hepatoma cells. Also included are various commercial approaches involving “lipofection” technology.

In certain embodiments of the invention, the liposome may be complexed with a hemagglutinating virus (HVJ), which has been shown to facilitate fusion with the cell membrane and promote cell entry (Kaneda et al., 1989). The liposome may be complexed or employed in conjunction with nuclear nonhistone chromosomal proteins (HMG-1) (Kato et al., 1991). In that such expression constructs have been successfully employed in transfer and expression of nucleic acid in vitro, then they are applicable for the present invention.

In certain embodiments of the present invention, the temperature at which infection of the host cells is performed is 37° C. However, in other embodiments, the infection temperature is at temperature that is less than 37° C. This is based on the inventors' discovery that infection temperatures less than 37° C. provide for optimal production of adenovirus. Thus, for example, the temperature may be at least about, at most about, or about 32.1° C., 32.2° C., 32.3° C., 32.4° C., 32.5° C., 32.6° C., 32.7° C., 32.8° C., 32.9° C., 33.0° C., 33.1° C., 33.2° C., 33.3° C., 33.4° C., 33.5° C., 33.6° C., 33.7° C., 33.8° C., 33.9° C., 34.0° C., 34.1° C. 34.2° C., 34.3° C., 34.4° C., 34.5° C., 34.6° C., 34.7° C., 34.8° C., 34.9° C., 35.0° C., 35.1° C., 35.2° C., 35.3° C., 35.4° C., 35.5° C., 35.6° C., 35.7° C., 35.8° C., 35.9° C., 36.0° C., 36.1° C., 36.2° C., 36.3° C., 36.4° C., 36.5° C., 36.6° C., 36.7° C., 36.8° C., and 36.9° C. and any range of temperature or increments of temperature derivable therein. Any method known to those of skill in the art may be used to measure the temperature of the cell culture media. One of skill in the art would be familiar with the wide range of methods available for measuring the temperature of culture media. One convenient way to measure temperature would be to use a real time digital device to measure the temperature inside an incubator.

In certain embodiments of the present invention, the methods for producing an adenovirus may involve initiating virus infection by diluting the host cells with fresh media and adenovirus. This avoids the need for a separate medium exchange step prior to infection. The invention contemplates that any amount of dilution of the host cells is contemplated by the present invention. In a certain embodiment, the host cells are diluted 10-fold with fresh media. The invention also contemplates any amount of virus added to initiate infection. However, in a certain embodiment of the present invention, virus infection will be initiated by adding 50 vp/host cell.

The embodiments of the present invention contemplate that virus infection can be allowed to proceed for various lengths of time. However, in a certain embodiments, virus infection is allowed to proceed for 1, 2, 3, to 4 days. In another embodiment of the present invention, host cell growth is allowed to occur in one bioreactor, and infection of host cells is conducted in a second bioreactor.

The term “adenovirus preparation” will be used herein to describe the reaction mixture following initiation of infection with adenovirus. The adenovirus preparation may include host cells that have undergone lysis, cell fragments, adenovirus, media, and any other components present in the reaction mixture during infection. The adenovirus preparation may include intact host cells, depending on how long infection was allowed to proceed. Some or all of the host cells may have undergone cell lysis, with release of viral particles into the surrounding media. The present invention contemplates that the methods for producing an adenovirus, adenovirus isolation will occur at any time and by any means known to those of skill in the art following infection. For example, in one embodiment of the present invention, isolating the adenovirus from the adenovirus preparation occurs 4 days after viral infection is completed.

V. Engineering of Viral Vectors

In particular embodiments, a recombinant adenovirus is contemplated for the delivery of expression constructs. “Recombinant adenovirus,” “adenovirus vector” or “adenoviral expression vector” is meant to include those constructs containing adenovirus sequences sufficient to (a) support packaging of the construct and (b) to ultimately express an expression construct cloned therein. The recombinant adenovirus may encode a recombinant gene. Thus, a recombinant adenovirus may include any of the engineered vectors that comprise adenoviral sequences.

An adenovirus expression vector according to the present invention comprises a genetically engineered form of the adenovirus. The nature of the adenovirus vector is not believed to be crucial to the successful practice of the invention. The adenovirus may be of any of the known serotypes and/or subgroups A-F. Adenovirus type 5 of subgroup C is one starting material in order to obtain one adenovirus vector for use in the present invention. This is because adenovirus type 5 is a human adenovirus about which a great deal of biochemical and genetic information is known, and it has historically been used for most constructions employing adenovirus as a vector.

Advantages of adenoviral gene transfer include the ability to infect a wide variety of cell types, ease of manipulation, high infectivity, and they can be grown to high titers (Wilson, 1996). Adenoviruses also are structurally stable (Marienfeld et al., 1999) and no genome rearrangement has been detected after extensive amplification (Parks et al., 1997; Brett et al., 1993).

Certain embodiments of the present invention pertain to methods of producing an adenovirus that involve replication-deficient adenovirus. Common approaches for generating adenoviruses for use as a gene transfer vector can be found in Graham and Prevec (1995) and U.S. Pat. Nos. 5,670,488, 5,824,544 and 5,932,210, for example.

A. Viral Vectors Encoding Therapeutic Genes.

In certain embodiments, the invention may include methods of producing an adenovirus where the adenovirus is a recombinant adenovirus encoding a recombinant gene. The recombinant gene may be operatively linked to a promoter. In certain other embodiments, the recombinant gene is a therapeutic gene. The invention contemplates use of any gene that has therapeutic or potential therapeutic value in the treatment of a disease or genetic disorder. One of skill in the art would be familiar with the wide range of such genes that have been identified.

The therapeutic genes involved may be those that encode proteins, structural or enzymatic RNAs, inhibitory products such as antisense RNA or DNA, or any other gene product. Expression is the generation of such a gene product or the resultant effects of the generation of such a gene product. Thus, enhanced expression includes the greater production of any therapeutic gene or the augmentation of that product's role in determining the condition of the cell, tissue, organ, or organism.

Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Hermens and Verhaagen, 1998), coronary diseases (Feldman et al., 1996), muscular diseases (Petrof, 1998), and various cancers such as colorectal (Dorai et al., 1999), bladder (Irie et al., 1999), prostate (Mincheff et al., 2000), head and neck (Blackwell et al., 1999), breast (Stewart et al., 1999), lung (Batra et al., 1999) and ovarian (Vanderkwaak et al., 1999).

The particular therapeutic gene encoded by the adenoviral vector is not limiting and includes those useful for various therapeutic and research purposes, as well as reporter genes and reporter gene systems and constructs useful in tracking the expression of transgenes and the effectiveness of adenoviral and adenoviral vector transduction. Thus, by way of example, the following are classes of possible genes whose expression may be enhanced by using the compositions and methods of the present invention: developmental genes (e.g., adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), oncogenes (e.g., ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES), tumor suppresser genes (e.g., APC, BRCA1, BRCA2, MADH4, MCC, NF1, NF2, RB1, TP53 and WT1), enzymes (e.g., ACP desaturases and hycroxylases, ADP-glucose pyrophorylases, ATPases, alcohol dehydrogenases, amylases, amyloglucosidases, catalases, cellulases, cyclooxygenases, decarboxylases, dextrinases, esterases, DNA and RNA polymerases, hyaluron synthases, galactosidases, glucanases, glucose oxidases, GTPases, helicases, hemicellulases, hyaluronidases, integrases, invertases, isomerases, kinases, lactases, lipases, lipoxygenases, lyases, lysozymes, pectinesterases, peroxidases, phosphatases, phospholipases, phophorylases, polygalacturonases, proteinases and peptideases, pullanases, recombinases, reverse transcriptases, topoisomerases, xylanases), reporter genes (e.g., Green fluorescent protein and its many color variants, luciferase, CAT reporter systems, β-galactosidase, etc.), blood derivatives, hormones, lymphokines (including interleukins), interferons, TNF, growth factors, neurotransmitters or their precursors or synthetic enzymes, trophic factors (such as BDNF, CNTF, NGF, IGF, GMF, aFGF, bFGF, NT3, NT5, and the like), apolipoproteins (such as ApoAI, ApoAIV, ApoE, and the like), dystrophin or a minidystrophic, tumor suppressor genes (such as p53, Rb, Rap1A, DCC, k-rev, and the like), genes coding for factors involved in coagulation (such as factors VII, VIII, IX, and the like), suicide genes (such as thymidine kinase), cytosine deaminase, or all or part of a natural or artificial immunoglobulin (Fab, ScFv, and the like). Other examples of therapeutic genes include fus, interferon α, interferon β, interferon γ, ADP (adenoviral death protein).

The therapeutic gene can also be an antisense gene or sequence whose expression in the target cell enables the expression of cellular genes or the transcription of cellular mRNA to be controlled, or instance ribozymes. Such sequence can, for example, be transcribed in the target cell into RNAs complementary to cellular mRNAs. The therapeutic gene can also be a gene coding for an antigenic peptide capable of generating an immune response in man. In this particular embodiment, the invention hence makes it possible to produce vaccines enabling humans to be immunized, in particular against microorganisms and viruses.

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

Other tumor suppressors that may be employed according to the present invention include BRCA1, BRCA2, zac1, p73, MMAC-1, ATM, HIC-1, DPC-4, FHIT, NF2, APC, DCC, PTEN, ING1, NOEY1, NOEY2, PML, OVCA1, MADR2, WT1, 53BP2, and IRF-1. Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p57 p27/p16 fusions, p21/p27 fusions, anti-thrombotic genes (e.g., COX-1, TFPI), PGS, Dp, E2F, ras, myc, neu, raf, erb,fms, trk, ret, gsp, hst, abl, E1A, p300, genes involved in angiogenesis (e.g., VEGF, FGF, thrombospondin, BAI-1, GDAIF, or their receptors) and MCC. Inducers of apoptosis, such as Bax, Bak, Bcl-X.s, Bik, Bid, Harakiri, Ad E1B, Bad and ICE-CED3 proteases, similarly could find use according to the present invention.

In certain embodiments the adenovirus comprises an exogenous gene construct that is an mda-7 gene. MDA-7 is another putative tumor suppressor that has been shown to suppress the growth of cancer cells that are p53-wild-type, p53-null, and p53-mutant. Also, the observed upregulation of the apoptosis-related Bax gene in p53 null cells indicates that MDA-7 is capable of using p53-independent mechanisms to induce the destruction of cancer cells.

Various genes encoding enzymes are also considered therapeutic genes. Particularly appropriate genes for expression include those genes that are thought to be expressed at less than normal level in the target cells of the subject mammal. Examples of particularly useful gene products include carbamoyl synthetase I, ornithine transcarbamylase, arginosuccinate synthetase, arginosuccinate lyase, and arginase. Other desirable gene products include fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, and Wilson's disease copper-transporting ATPase. Other examples of gene products include cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase and human thymidine kinase. Hormones are another group of genes that may be used in the vectors described herein. Included are growth hormone, prolactin, placental lactogen, luteinizing hormone, follicle-stimulating hormone, chorionic gonadotropin, thyroid-stimulating hormone, leptin, adrenocorticotropin (ACTH), angiotensin I and II, β-endorphin, β-melanocyte stimulating hormone (β-MSH), cholecystokinin, endothelin I, galanin, gastric inhibitory peptide (GIP), glucagon, insulin, lipotropins, neurophysins, somatostatin, calcitonin, calcitonin gene related peptide (CGRP), β-calcitonin gene related peptide, hypercalcemia of malignancy factor (1-40), parathyroid hormone-related protein (107-139) (PTH-rP), parathyroid hormone-related protein (107-111) (PTH-rP), glucagon-like peptide (GLP-1), pancreastatin, pancreatic peptide, peptide YY, PHM, secretin, vasoactive intestinal peptide (VIP), oxytocin, vasopressin (AVP), vasotocin, enkephalinamide, metorphinamide, alpha melanocyte stimulating hormone (α-MSH), atrial natriuretic factor (5-28) (ANF), amylin, amyloid P component (SAP-1), corticotropin releasing hormone (CRH), growth hormone releasing factor (GHRH), luteinizing hormone-releasing hormone (LHRH), neuropeptide Y, substance K (neurokinin A ), substance P and thyrotropin releasing hormone (TRH). Other classes of genes that are contemplated to be inserted into the vectors of the present invention include interleukins and cytokines. Interleukin 1 (IL-1), IL-2, IL-3, IL-4, IL-5, IL-6, IL-7, IL-8, IL-9, IL-10, IL-11 IL-12, GM-CSF and G-CSF.

Examples of diseases for which the present viral vector would be useful include, but are not limited to, adenosine deaminase deficiency, human blood clotting factor IX deficiency in hemophilia B, and cystic fibrosis, which would involve the replacement of the cystic fibrosis conductance regulator gene. The vectors embodied in the present invention could also be used for treatment of hyperproliferative disorders such as rheumatoid arthritis or restenosis by transfer of genes encoding angiogenesis inhibitors or cell cycle inhibitors. Transfer of prodrug activators such as the HSV-TK gene can be also be used in the treatment of hyperploiferative disorders, including cancer.

1. Antisense Constructs.

Oncogenes such as ras, myc, neu, raf, erb, src, fms, jun, trk, ret, gsp, hst, bcl and abl also are suitable targets. However, for therapeutic benefit, these oncogenes would be expressed as an antisense nucleic acid, so as to inhibit the expression of the oncogene. The term “antisense nucleic acid” is intended to refer to the oligonucleotides complementary to the base sequences of oncogene-encoding DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

As an alternative to targeted antisense delivery, targeted ribozymes may be used. The term “ribozyme” refers to an RNA-based enzyme capable of targeting and cleaving particular base sequences in oncogene DNA and RNA. Ribozymes can either be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

2. Antigens for Vaccines

Other therapeutic genes might include genes encoding antigens such as viral antigens, bacterial antigens, fungal antigens or parasitic antigens. Viruses include picomavirus, coronavirus, togavirus, flavirvirus, rhabdovirus, paramyxovirus, orthomyxovirus, bunyavirus, arenvirus, reovirus, retrovirus, papovavirus, parvovirus, herpesvirus, poxvirus, hepadnavirus, and spongiform virus. Viral targets include influenza, herpes simplex virus 1 and 2, measles, small pox, polio or HIV. Pathogens include trypanosomes, tapeworms, roundworms, helminths, Also, tumor markers, such as fetal antigen or prostate specific antigen, may be targeted in this manner. Examples include HIV env proteins and hepatitis B surface antigen. Administration of a vector according to the present invention for vaccination purposes would require that the vector-associated antigens be sufficiently non-immunogenic to enable long term expression of the transgene, for which a strong immune response would be desired. Typically, vaccination of an individual would only be required infrequently, such as yearly or biennially, and provide long term immunologic protection against the infectious agent.

B. Control Regions

In order for the viral vector to effect expression of a transcript encoding a therapeutic gene, the polynucleotide encoding the therapeutic gene will be under the transcriptional control of a promoter and a polyadenylation signal. Therefore, certain embodiments of the present invention involve methods for producing an adenovirus wherein the adenovirus comprises an adenoviral vector encoding an exogenous gene construct that is operatively linked to a promoter. A “promoter” refers to a DNA sequence recognized by the synthetic machinery of the host cell, or introduced synthetic machinery, that is required to initiate the specific transcription of a gene. A polyadenylation signal refers to a DNA sequence recognized by the synthetic machinery of the host cell, or introduced synthetic machinery, that is required to direct the addition of a series of nucleotides on the end of the mRNA transcript for proper processing and trafficking of the transcript out of the nucleus into the cytoplasm for translation. The phrases “operatively linked,” “under control,” and “under transcriptional control” mean that the promoter is in the correct location in relation to the polynucleotide to control RNA polymerase initiation and expression of the polynucleotide.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter that is employed to control the expression of a therapeutic gene is not believed to be critical, so long as it is capable of expressing the polynucleotide in the targeted cell. The promoter may be a tissue-specific promoter or an inducible promoter. Examples of promoters that may be employed include SV40 EI, RSV LTR, β-actin, CMV-IE, adenovirus major late, polyoma F9-1, α-fetal protein promoter, egr-1, or tyrosinase promoter. One of skill in the art would be familiar with the range of options available for promoters that can be used to control the expression of a therapeutic gene. Thus, where a human cell is targeted, it is preferable to position the polynucleotide coding region adjacent to and under the control of a promoter that is capable of being expressed in a human cell. Generally speaking, such a promoter might include either a human or viral promoter. A list of promoters includes, but is not limited to Immunoglobulin Heavy Chain, Immunoglobulin Light Chain, T-Cell Receptor, HLA DQ a and DQ β, β-Interferon, Interleukin-2, Interleukin-2 Receptor, MHC Class II 5, MHC Class II HLA-DRα, β-Actin, Muscle Creatine Kinase, Prealbumin (Transthyretin), Elastase I, Metallothionein, Collagenase, Albumin Gene, α-Fetoprotein, τ-Globin, β-Globin, c-fos, c-HA-ras, Insulin, Neural Cell Adhesion Molecule (NCAM), α-Antitrypsin, H2B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular Dystrophy, SV40, Polyoma, Retroviruses, Papilloma Virus, Hepatitis B Virus, Human Immunodeficiency Virus, Cytomegalovirus, or Gibbon Ape Leukemia Virus promoters and the like.

The promoter may be a constitutive promoter, an inducible promoter, or a tissue-specific promoter. An inducible promoter is a promoter which is inactive or exhibits low activity except in the presence of an inducer substance. Some examples of promoters that may be included as a part of the present invention include, but are not limited to, MT II, MMTV, Collagenase, Stromelysin, SV40, Murine MX gene, α-2-Macroglobulin, MHC class I gene h-2kb, HSP70, Proliferin, Tumor Necrosis Factor, or Thyroid Stimulating Hormone α gene. It is understood that any inducible promoter may be used in the practice of the present invention and that all such promoters would fall within the spirit and scope of the claimed invention. A promoter that is “endogenous” or “constitutive” is a promoter that is one naturally associated with a gene or sequence, as may be obtained by isolating the 5′ non-coding sequences located upstream of the coding segment and/or exon. Promoters and their inducers include, but are not limited to (element/inducer) MT II/Phorbol Ester (TPA) Heavy metals, MMTV (mouse mammary tumor virus)/Glucocorticoids, β-Interferon/poly(rI)X/poly(rc), Adenovirus 5 E2/E1a, c-jun/Phorbol Ester (TPA), H₂O₂, Collagenase/Phorbol Ester (TPA), Stromelysin/Phorbol Ester (TPA), IL-1, SV40/Phorbol Ester (TPA), Murine MX Gene/Interferon, Newcastle Disease Virus, GRP78 Gene/A23187, α-2-Macroglobulin/IL-6, Vimentin/Serum, MHC Class I Gene H-2kB/Interferon, HSP70/E1a, SV40 Large T Antigen, Proliferin/Phorbol Ester-TPA, Tumor Necrosis Factor/FMA, or Thyroid Stimulating Hormone a Gene/Thyroid Hormone.

In various embodiments, the human cytomegalovirus (CMV) immediate early gene promoter, the SV40 early promoter, or the Rous sarcoma virus long terminal repeat can be used to obtain high-level expression of the polynucleotide of interest. The use of other viral or mammalian cellular or bacterial phage promoters which are well-known in the art to achieve expression of polynucleotides is contemplated as well, provided that the levels of expression are sufficient to produce a growth inhibitory effect.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base (EPDB)) could also be used to drive expression of a particular construct. Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed. Such polyadenylation signals as that from SV40, bovine growth hormone, and the herpes simplex virus thymidine kinase gene have been found to function well in a number of target cells.

VI. Methods of Isolation Adenovirus

Adenoviral infection results in the lysis of the cells being infected. The lytic characteristics of adenovirus infection permit two different modes of virus isolation and production. One is harvesting infected cells prior to cell lysis. The other mode is harvesting virus supernatant after complete cell lysis by the produced virus. For the latter mode, longer incubation times are required in order to achieve complete cell lysis. This prolonged incubation time after virus infection creates a serious concern about increased possibility of generation of replication competent adenovirus (RCA), particularly for the current first generation adenoviral vectors (E1-deleted vector). Therefore, in certain embodiments of the present invention, the methods for producing an adenovirus involve harvesting the host cells and then lysing the host cells. Table 6 lists the most common methods that have been used for lysing cells after cell harvest.

TABLE 6
Exemplary methods used for cell lysis

| Methods | Procedures | Comments |
|---|---|---|
| Freeze-thaw | Cycling between dry ice and | Easy to carry out at lab |
| | 37° C. water bath | scale. High cell lysis |
| | | efficiency |
| | | Not scaleable |
| | | Not recommended for large |
| | | scale manufacturing |
| Solid Shear | French Press | Capital equipment |
| | Hughes Press | investment |
| | | Virus containment concerns |
| | | Lack of experience |
| Detergent | Non-ionic detergent solutions | Easy to carry out at both |
| lysis | such as Tween ®, Triton ®, | lab and manufacturing scale |
| | NP-40, etc. | Wide variety of detergent |
| | | choices |
| | | Concerns of residual |
| | | detergent in finished |
| | | product |
| Hypotonic | water, citric buffer | Low lysis efficiency |
| solution lysis | | |
| Liquid Shear | Homogenizer | Capital equipment |
| | Impinging Jet | investment |
| | Microfluidizer | Virus containment concerns |
| | | Scalability concerns |
| Sonication | Ultrasound | Capital equipment |
| | | investment |
| | | Virus containment concerns |
| | | Noise pollution |
| | | Scalability concern |

A. Detergents

In certain embodiments of the present invention, the methods for producing an adenovirus involve isolating the adenovirus by lysing the host cells with a detergent. Cells are bounded by membranes. In order to release components of the cell, it is necessary to break open the cells. The most advantageous way in which this can be accomplished, according to the present invention, is to solubilize the membranes with the use of detergents. Detergents are amphipathic molecules with an apolar end of aliphatic or aromatic nature and a polar end which may be charged or uncharged. Detergents are more hydrophilic than lipids and thus have greater water solubility than lipids. They allow for the dispersion of water insoluble compounds into aqueous media and are used to isolate and purify proteins in a native form.

Any detergent capable of lysing the host cells is contemplated by the claimed invention. One of skill in the art would be familiar with the wide range of detergents available for lysing cells. Detergents can be denaturing or non-denaturing. The former can be anionic such as sodium dodecyl sulfate or cationic such as ethyl trimethyl ammonium bromide. These detergents totally disrupt membranes and denature the protein by breaking protein-protein interactions. Non denaturing detergents can be divided into non-anionic detergents such as Triton® X-100, bile salts such as cholates and zwitterionic detergents such as CHAPS. Zwitterionics contain both cationic and anion groups in the same molecule, the positive electric charge is neutralized by the negative charge on the same or adjacent molecule.

Denaturing agents such as SDS bind to proteins as monomers and the reaction is equilibrium driven until saturated. Thus, the free concentration of monomers determines the necessary detergent concentration. SDS binding is cooperative i.e. the binding of one molecule of SDS increase the probability of another molecule binding to that protein, and alters proteins into rods whose length is proportional to their molecular weight.

Non-denaturing agents such as Triton® X-100 do not bind to native conformations nor do they have a cooperative binding mechanism. These detergents have rigid and bulky apolar moieties that do not penetrate into water soluble proteins. They bind to the hydrophobic parts of proteins. Triton® X100 and other polyoxyethylene nonanionic detergents are inefficient in breaking protein-protein interaction and can cause art factual aggregations of protein. These detergents will, however, disrupt protein-lipid interactions but are much gentler and capable of maintaining the native form and functional capabilities of the proteins.

Detergent removal can be attempted in a number of ways. Dialysis works well with detergents that exist as monomers. Dialysis is somewhat ineffective with detergents that readily aggregate to form micelles because the micelles are too large to pass through dialysis. Ion exchange chromatography can be utilized to circumvent this problem. The disrupted protein solution is applied to an ion exchange chromatography column and the column is then washed with buffer minus detergent. The detergent will be removed as a result of the equilibration of the buffer with the detergent solution. Alternatively the protein solution may be passed through a density gradient. As the protein sediments through the gradients the detergent will come off due to the chemical potential.

Often a single detergent is not versatile enough for the solubilization and analysis of the milieu of proteins found in a cell. The proteins can be solubilized in one detergent and then placed in another suitable detergent for protein analysis. The protein detergent micelles formed in the first step should separate from pure detergent micelles. When these are added to an excess of the detergent for analysis, the protein is found in micelles with both detergents. Separation of the detergent-protein micelles can be accomplished with ion exchange or gel filtration chromatography, dialysis or buoyant density type separations.

1. Triton® X-Detergents

This family of detergents (Triton® X-100, X114 and NP-40) have the same basic characteristics but are different in their specific hydrophobic-hydrophilic nature. All of these heterogeneous detergents have a branched 8-carbon chain attached to an aromatic ring. This portion of the molecule contributes most of the hydrophobic nature of the detergent. Triton® X detergents are used to solublize membrane proteins under non-denaturing conditions. The choice of detergent to solubilize proteins will depend on the hydrophobic nature of the protein to be solubilized. Hydrophobic proteins require hydrophobic detergents to effectively solubilize them.

Triton® X-100 and NP-40 are very similar in structure and hydrophobicity and are interchangeable in most applications including cell lysis, delipidation protein dissociation and membrane protein and lipid solubilization. Generally 2 mg of detergent is used to solubilize 1 mg membrane protein or 10 mg detergent/i mg of lipid membrane. Triton® X-114 is useful for separating hydrophobic from hydrophilic proteins.

2. Brij® Detergents

These are similar in structure to Triton® X detergents in that they have varying lengths of polyoxyethylene chains attached to a hydrophobic chain. However, unlike Triton® X detergents, the Brij® detergents do not have an aromatic ring and the length of the carbon chains can vary. The Brij® detergents are difficult to remove from solution using dialysis but may be removed by detergent removing gels. Brij® 58 is most similar to Triton® X100 in its hydrophobic/hydrophilic characteristics. Brij®-35 is a commonly used detergent in HPLC applications.

3. Dializable Nonionic Detergents

η-Octyl-β-D-glucoside (octylglucopyranoside) and η-Octyl-β-D-thioglucoside (octylthioglucopyranoside, OTG) are nondenaturing nonionic detergents which are easily dialyzed from solution. These detergents are useful for solubilizing membrane proteins and have low UV absorbances at 280 nm. Octylglucoside has a high CMC of 23-25 mM and has been used at concentrations of 1.1-1.2% to solubilize membrane proteins.

Octylthioglucoside was first synthesized to offer an alternative to octylglucoside. Octylglucoside is expensive to manufacture and there are some inherent problems in biological systems because it can be hydrolyzed by β-glucosidase.

4. Tween® Detergents

The Tween® detergents are nondenaturing, nonionic detergents. They are polyoxyethylenesorbitan esters of fatty acids. Tween® 20 and Tween® 80 detergents are used as blocking agents in biochemical applications and are usually added to protein solutions to prevent nonspecific binding to hydrophobic materials such as plastics or nitrocellulose. They have been used as blocking agents in ELISA and blotting applications. Generally, these detergents are used at concentrations of 0.01-1.0% to prevent nonspecific binding to hydrophobic materials.

Tween® 20 and other nonionic detergents have been shown to remove some proteins from the surface of nitrocellulose. Tween® 80 has been used to solubilize membrane proteins, present nonspecific binding of protein to multiwell plastic tissue culture plates and to reduce nonspecific binding by serum proteins and biotinylated protein A to polystyrene plates in ELISA.

The difference between these detergents is the length of the fatty acid chain. Tween® 80 is derived from oleic acid with a C18 chain while Tween® 20 is derived from lauric acid with a C12 chain. The longer fatty acid chain makes the Tween® 80 detergent less hydrophilic than Tween® 20 detergent. Both detergents are very soluble in water.

The Tween® detergents are difficult to remove from solution by dialysis, but Tween® 20 can be removed by detergent removing gels. The polyoxyethylene chain found in these detergents makes them subject to oxidation (peroxide formation) as is true with the Triton® X and Brij® series detergents.

5. Zwitterionic Detergents

The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). This detergent has low absorbances at 280 nm making it useful when protein monitoring at this wavelength is necessary. CHAPS is compatible with the BCA Protein Assay and can be removed from solution by detergent removing gel. Proteins can be iodinated in the presence of CHAPS

CHAPS has been successfully used to solubilize intrinsic membrane proteins and receptors and maintain the functional capability of the protein. When cytochrome P-450 is solubilized in either Triton® X-100 or sodium cholate aggregates are formed.

B. Non-Detergent Methods

Various non-detergent methods may be employed in conjunction with other advantageous aspects of the present invention:

1. Freeze-Thaw

Freeze-thaw has been a widely used technique for lysis cells in a gentle and effective manner. Cells are generally frozen rapidly in, for example, a dry ice/ethanol bath until completely frozen, then transferred to a 37° C. bath until completely thawed. This cycle is repeated a number of times to achieve complete cell lysis.

2. Sonication

High frequency ultrasonic oscillations have been found to be useful for cell disruption. The method by which ultrasonic waves break cells is not fully understood but it is known that high transient pressures are produced when suspensions are subjected to ultrasonic vibration. The main disadvantage with this technique is that considerable amounts of heat are generated. In order to minimize heat effects specifically designed glass vessels are used to hold the cell suspension. Such designs allow the suspension to circulate away from the ultrasonic probe to the outside of the vessel where it is cooled as the flask is suspended in ice.

3. High Pressure Extrusion

High pressure extrusion is a frequently used method to disrupt microbial cells. The French pressure cell employs pressures of 10.4 10⁷ Pa (16,000 p.s.i.) to break cells open. These apparatus consists of a stainless steel chamber which opens to the outside by means of a needle valve. The cell suspension is placed in the chamber with the needle valve in the closed position. After inverting the chamber, the valve is opened and the piston pushed in to force out any air in the chamber. With the valve in the closed position, the chamber is restored to its original position, placed on a solid based and the required pressure is exerted on the piston by a hydraulic press. When the pressure has been attained the needle valve is opened fractionally to slightly release the pressure and as the cells expand they burst. The valve is kept open while the pressure is maintained so that there is a trickle of ruptured cell which may be collected.

4. Solid Shear Methods

Mechanical shearing with abrasives may be achieved in Mickle shakers which oscillate suspension vigorously (300-3000 time/min) in the presence of glass beads of 500 nm diameter. This method may result in organelle damage. A more controlled method is to use a Hughes press where a piston forces most cells together with abrasives or deep frozen paste of cells through a 0.25 mm diameter slot in the pressure chamber. Pressures of up to 5.5×10⁷Pa (8000 p.s.i.) may be used to lyse bacterial preparations.

5. Liquid Shear Methods

These methods employ blenders, which use high speed reciprocating or rotating blades, homogenizers which use an upward/downward motion of a plunger and ball and microfluidizers or impinging jets which use high velocity passage through small diameter tubes or high velocity impingement of two fluid streams. The blades of blenders are inclined at different angles to permit efficient mixing. Homogenizers are usually operated in short high speed bursts of a few seconds to minimize local heat. These techniques are not generally suitable for microbial cells but even very gentle liquid shear is usually adequate to disrupt animal cells.

6. Hypotonic/Hypertonic Methods

Cells are exposed to a solution with a much lower (hypotonic) or higher (hypertonic) solute concentration. The difference in solute concentration creates an osmotic pressure gradient. The resulting flow of water into the cell in a hypotonic environment causes the cells to swell and burst. The flow of water out of the cell in a hypertonic environment causes the cells to shrink and subsequently burst.

VII. Methods of Concentration and Foltration

The present invention involve methods of producing an adenovirus that involve isolating the adenovirus. Methods of isolating the adenovirus from host cells include, for example, clarification, concentration, and diafiltration. One step in the purification process can include clarification of the cell lysate to remove large particulate matter, particularly cellular components, from the cell lysate. Clarification of the lysate can be achieved using a depth filter or by tangential flow filtration. In one embodiment of the present invention, the cell lysate is concentrated. Concentrating the crude cell lysate may include any step known to those of skill in the art. For example, the crude cell lysate may be passed through a depth filter, which consists of a packed column of relatively non-adsorbent material (e.g. polyester resins, sand, diatomeceous earth, colloids, gels, and the like). In tangential flow filtration (TFF), the lysate solution flows across a membrane surface which facilitates back diffusion of solute from the membrane surface into the bulk solution. Membranes are generally arranged within various types of filter apparatus including open channel plate and frame, hollow fibers, and tubules.

After clarification and prefiltration of the cell lysate, the resultant virus supernatant may be concentrated and buffer may be exchanged by diafiltration. The virus supernatant can be concentrated by tangential flow filtration across an ultrafiltration membrane of 100-300K nominal molecular weight cutoff. Ultrafiltration is a pressure-modified convective process that uses semi-permeable membranes to separate species by molecular size, shape, and/or charge. It separates solvents from solutes of various sizes, independent of solute molecular size. Ultrafiltration is gentle, efficient and can be used to simultaneously concentrate and desalt solutions. Ultrafiltration membranes generally have two distinct layers: a thin (0.1-1.5 μm), dense skin with a pore diameter of 10-400 angstroms and an open substructure of progressively larger voids which are largely open to the permeate side of the ultrafilter. Any species capable of passing through the pores of the skin can therefore freely pass through the membrane. For maximum retention of solute, a membrane is selected that has a nominal molecular weight cut-off well below that of the species being retained. In macromolecular concentration, the membrane enriches the content of the desired biological species and provides filtrate cleared of retained substances. Microsolutes are removed convectively with the solvent. As concentration of the retained solute increases, the ultrafiltration rate diminishes.

Some embodiments of the present invention involve use of exchanging buffer of the crude cell lysate. Buffer exchange, or diafiltration, involves using ultrafilters is an ideal way for removal and exchange of salts, sugars, non-aqueous solvents separation of free from bound species, removal of material of low molecular weight, or rapid change of ionic and pH environments. Microsolutes are removed most efficiently by adding solvent to the solution being ultrafiltered at a rate equal to the ultrafiltration rate. This washes microspecies from the solution at constant volume, purifying the retained species.

A. Removing Nucleic Acid Contaminants

Certain embodiments of the methods for producing an adenovirus involve reducing the concentration of contaminating nucleic acids in a crude cell lysate. The present invention employs nucleases to remove contaminating nucleic acids. Exemplary nucleases include Benzonase®, Pulmozyme®; or any other DNase or RNase commonly used within the art.

Enzymes such as Benzonaze® degrade nucleic acid and have no proteolytic activity. The ability of Benzonase® to rapidly hydrolyze nucleic acids makes the enzyme ideal for reducing cell lysate viscosity. It is well known that nucleic acids may adhere to cell derived particles such as viruses. The adhesion may interfere with separation due to agglomeration, change in size of the particle or change in particle charge, resulting in little if any product being recovered with a given purification scheme. Benzonase® is well suited for reducing the nucleic acid load during purification, thus eliminating the interference and improving yield.

As with all endonucleases, Benzonase® hydrolyzes internal phosphodiester bonds between specific nucleotides. Upon complete digestion, all free nucleic acids present in solution are reduced to oligonucleotides 2 to 4 bases in length.

B. Size Partitioning Purification

According to one aspect of the invention it has been found that size partitioning purification techniques may be used to provide adenoviral preparations of sufficient purity that they may be therapeutically administered without additional purification steps such as chromatography and other methods previously considered necessary. Without intending to be bound by any particular theory of the invention it is believed that the steps of processing viral host cells in a cell suspension culture in a serum free media results in a viral particle product with a reduced load of contaminants. Moreover, the contaminants are of a size and nature that they may be readily separated from viral particles by a simple size partitioning purification step.

Membrane filtration is a well known technique in the art of bioprocessing. A membrane is defined as a structure having lateral dimensions much greater than its thickness, through which mass transfer may occur under a variety of driving forces. While many filters may be considered membranes, filters also include materials whose lateral dimensions are not usually 100 times greater than their depth and whose separation function is primarily by capture of species or particles through their depth. The most common parameters used to characterize membranes fall in three general categories. These are transport properties, pore (geometric) characteristics, and surface (or predominantely chemical) properties. Nevertheless, the transport properties depend significantly upon the pore and surface characteristics. While membrane separation can be slower and a lower volume process than other separation processes, its effectiveness makes it a method that can be used for retrieving small amounts of valuable products.

Membrane filter systems may be designed in a variety of manners to have different filtration properties. Design criteria include: operation in dead-end (with or without stirring) or cross flow mode; full or partial recovery of the feed mixture; application of an external transmembrane pressure via pumping, inert gas blanket, or evacuation of the permeate side of the device; and the use of flat sheets (either singly or multiply), hollow fiber bundle, or tubular membranes. Size partitioning separation methods utilize a size partitioning membrane which may be a dialysis or other similar membrane as would be known to those of ordinary skill in the art. Suitable dialysis membrane materials useful in the size partitioning membrane filtration of the invention include those commercially available such as those produced from polyethersulphone, polycarbonate, nylon, polypropylene, and the like. Suppliers of these dialysis membrane materials include Akzo-Nobel, Millipore, Inc., Poretics, Inc., and Pall Corp., by way of example. Size partitioning membranes having pore sizes of less than 0.08 microns are useful in practice of the invention with those having pore sizes less than 0.05 microns and less than 0.02 microns and greater than 0.001 microns can be used. Such membranes are capable of allowing the passage of desired viral particles while retaining undesired contaminants.

According to one aspect of the invention, tangential flow filtration (TFF) units, also known as “cross-flow filtration,” have been found to be particularly advantageous for practice of the invention. Tangential flow filtration is a pressure driven separation process wherein fluid is pumped tangentially long the surface of a membrane. An applied pressure serves to force a portion of the fluid including contaminants through the membrane to the filtrate size. Particulates and macromolecules that are too large to pass through the membrane pores are retained on the upstream side. In contrast to normal flow filtration (NFF) techniques in which the retained components build up on the surface of the membrane, tangential flow filtration sweeps the retained components along by the flow of the fluid.

TFF is classified based on the size of components being separated. A membrane pore size rating is typically given as a micron value and indicates that particles larger than the rating will be retained by the membrane. A nominal molecular weight limit (NMWL), on the other hand, is an indication that most dissolved macromolecules with molecular weights higher than the NMWL and some with molecular weights lower than the NMWL will be retained by the membrane. A component's shape, its ability to deform, and its interaction with other components in the solution all affect retention. Different membrane manufacturers use different criteria to assign the NMWL ratings to a family of membranes but those of ordinary skill would be able to determine the appropriate rating empirically.

Ultrafiltration is one of the most widely used forms of TFF and is used to separate proteins from buffer components for buffer exchange, desalting, or concentration but may also be used for Virus Filtration. Typical NMWL ratings for virus filtration range from 100 kD to 500 kD, or up to 0.05 to 0.08 microns.

Diafiltration is a TFF process than can be performed in combination with any of the other categories of separation to enhance either yield or purity. During diafiltration, buffer is introduced into the recycle tank while filtrate is removed from the unit operation. In processes where the product is in the retentate, diafiltration washes components out of the product pool into the filtrate, thereby exchanging buffers and reducing the concentration of undesirable species. When the product is in the filtrate, diafiltration washes it through the membrane into a collection vessel.

In TFF unit operation, a pump is used to generate flow of the feed stream through the channel between two membrane surfaces. During each pass of fluid over the surface of the membrane, the applied pressure forces a portion of the fluid through the membrane and into the filtrate stream. The result is a gradient in the feedstock concentration from the bulk conditions at the center of the channel to the more concentrated wall conditions at the membrane surface. There is also a concentration gradient along the length of the feed channel from the inlet to the outlet (retentate) at progressively more fluid passes to the filtrate side. The flow of feedstock along the length of the membrane causes a pressure drop from the feed to the retentate end of the channel. The flow on the filtrate side of the membrane is typically low and there is little restriction, so the pressure along the length of the membrane on the filtrate side is approximately constant.

Membranes may be fabricated from various materials offering alternatives in flushing characteristics and chemical compatibility. Suitable materials include cellulose, polyethersulfone and other materials known to those of skill in the art. In certain embodiments polyethersulfone is used. Typical polyethersulfone membranes tend to adsorb protein as well as other biological components, leading to membrane fouling and lowered flux. Some membranes are hydrophilically modified to be more resistant to fouling such as Biomax® (Millipore).

Those of skill in the art would recognize that various types of TFF modules would be useful in practice of the invention. Useful TFF modules include but are not limited to flat plate modules (also known as cassettes), spiral wound modules, and hollow fiber modules. In flat plate modules, layers of membrane either with or without alternating layers of separator screen are stacked together and then sealed into a package. Feed fluid is pumped into alternating channels at one end of the stack and the filtrate passes through the membrane into the filtrate channels. Flat plat modules generally have high packing densities (area of membrane surface per area of floor space), allow linear scaling, and some offer the choice of open or turbulence promoted channels.

Spiral wound modules comprise alternating layers of membrane and separator screen wound around a hollow central core the feed stream is pumped into one end and flows down the axis of the cartridge. Filtrate passes through the membrane and spirals to the core, where it is removed. The separator screens increase turbulence in the flowpath, leading to a higher efficiency module than hollow fibers. One drawback to spiral wound modules is that they are not linearly scaleable because either the feed flowpath length (cartridge length) or the filtrate flowpath length (cartridge width) must be changed within scales.

Hollow fiber modules comprise a bundle of membrane tubes with narrow diameters (typically in the range of 0.1 to 2.0 mm). In a hollow fiber module, the feed stream is pumped into the lumen (inside) of the tube and the filtrate passes through the membrane to the shell side, where it is removed. Because of the very open feed flowpath, low shear is generated even with moderate cross flow rates.

For any given module, key process parameters may then be readily optimized by those of ordinary skill. Such parameters include cross flow rate, transmembrane pressure (TMP), filtrate control, membrane area, and diafiltration design. Cross flow rate depends upon which module is selected. In general, a higher cross flow rate gives higher flux at equal TMP and increases the sweeping action across the membrane and reduces the concentration gradient towards the membrane surface. Many TFF applications apply a cross flow and pressure set point and the filtrate flows uncontrolled and unrestricted out of the module. This is the simplest type of operation but in some circumstances it might be desired to use some type of filtrate control beyond that achieved by simply adjusting the pressure with the retentate valve. Membrane area is selected after determining the process flow and the total volume to be processed and is also dependent upon process time.

According to one aspect of the invention a plate and frame TFF system was used with each of a 300 kD, a 500 kD or a 1000 kD polysulfone membrane having a surface area of 1.1 ft². The cross flow rate was 900 mL/ft²/min. and the transmembrane pressure was about 7 psi. The filtrate rate was not actively controlled and the diafiltration was performed using the consistent volume method.

The invention provides methods of producing purified adenovirus compositions which avoid the necessity of multiple purification steps including chromatographic purification steps. Nevertheless, additional purification steps including those known to the art may be practiced if desired. Such methods include those taught in U.S. Pat. No. 6,194,191, the disclosure of which is incorporated by reference, including density gradient centrifugation; chromatography including size exclusion chromatography, ion exchange chromatography, high performance liquid chromatography (HPLC), and the like.

VIII. Pharmaceutical Formulations

The present invention includes, in certain embodiments, methods formulating an adenovirus into a pharmaceutically acceptable composition. The present invention also includes compositions of adenovirus prepared by one of the processes disclosed in this application, wherein the composition is a pharmaceutically acceptable composition.

When purified according to the methods set forth in this application, the viral particles of the present invention will be administered to a subject or a cell with in vitro, ex vivo or in vivo administration being contemplated. Thus, it will be desirable to prepare the compositions as a pharmaceutical composition appropriate for the intended application. Generally this will entail preparing a pharmaceutical composition that is essentially free of pyrogens, as well as any other impurities that could be harmful to humans or animals. It may also be desired to employ appropriate salts and buffers to render the compositions and their components stable and allow for uptake by target cells.

The phrase “pharmaceutically acceptable composition” refers to molecular entities and compositions that do not produce an adverse, allergic, or other untoward reaction when administered to an animal, or a human, as appropriate. As used herein, “pharmaceutically acceptable composition” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the composition. In addition, the composition can include supplementary inactive ingredients. For instance, the composition for use as a mouthwash may include a flavorant or the composition may contain supplementary ingredients to make the formulation timed-release.

Aqueous compositions of the present invention comprise an effective amount of virus dissolved, or dispersed in a pharmaceutically acceptable carrier or aqueous medium. Such compositions also are referred to as inocula. Examples of aqueous compositions include a formulation for intravenous administration or a formulation for topical application.

Solutions of the compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The viral particles and compositions of the present invention may include classic pharmaceutical preparations for use in therapeutic regimens, including their administration to humans. Administration of therapeutic compositions according to the present invention will be via any common route so long as the target tissue or cell is available via that route. This includes oral, nasal, buccal, rectal, vaginal, or topical. Alternatively, administration may be by orthotopic, intradermal subcutaneous, intramuscular, intraperitoneal, or intravenous injection. Such compositions would normally be administered as pharmaceutically acceptable compositions that include physiologically acceptable carriers, buffers or other excipients. For application against tumors, direct intratumoral injection, inject of a resected tumor bed, regional (i.e., lymphatic) or general administration is contemplated. It also may be desired to perform continuous perfusion over hours or days via a catheter to a disease site, e.g., a tumor or tumor site.

The therapeutic and preventive compositions of the present invention are advantageously administered in the form of liquid solutions or suspensions; solid forms suitable for solution in, or suspension in, liquid prior to topical use may also be prepared. A typical composition for such purpose comprises a pharmaceutically acceptable carrier. For instance, the composition may contain 10 mg, 25 mg, 50 mg or up to about 100 mg of human serum albumin per ml of phosphate buffered saline. Other pharmaceutically acceptable carriers include aqueous solutions, non-toxic excipients, including salts, preservatives, buffers, anti-oxidants, and the like. Examples of non-aqueous solvents are propylene glycol, polyethylene glycol, vegetable oil, and injectable organic esters such as ethyloleate. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components of the pharmaceutical composition are adjusted according to well-known parameters.

Oral formulations include such normally employed excipients as, for example, pharmaceutical grades of mannitol, lactose, starch, magnesium stearate, sodium saccharine, cellulose, magnesium carbonate and the like. These compositions take the form of solutions such as mouthwashes and mouthrinses, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2, 20, 25, 40, 50 to about 50, 60, 70, 75% of the weight of the unit, and/or between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

For oral administration the expression cassette of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin, and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

One may also use solutions and/or sprays, hyposprays, aerosols and/or inhalants in the present invention for administration. One example is a spray for administration to the aerodigestive tract. The sprays are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Additional formulations which are suitable for other modes of administration include vaginal or rectal suppositories and/or pessaries. Formulations for other types of administration that is topical include, for example, a cream, suppository, ointment or salve.

An effective amount of the therapeutic agent is determined based on the intended goal, for example (i) inhibition of tumor cell proliferation, (ii) elimination or killing of tumor cells, (iii) vaccination, or (iv) gene transfer for long term expression of a therapeutic gene. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the result desired. Multiple gene therapeutic regimens are expected, especially for adenovirus.

In certain embodiments of the present invention, an adenoviral vector encoding a tumor suppressor gene will be used to treat cancer patients. Typical amounts of an adenovirus vector used in gene therapy of cancer is at least about, at most about, or about 10³-10¹⁵ PFU/dose, (10³, 10⁴, 10⁵, 10⁶, 10⁷, 10⁸, 10⁹, 10¹⁰, 10¹¹, 10¹², 10¹³, 10¹⁴, 10¹⁵ or more) wherein the dose may be divided into several injections at different sites within a solid tumor.

In another embodiment of the present invention, an adenoviral vector encoding a therapeutic gene may be used to vaccinate humans or other mammals. A typical dose would be from 10⁶ to 10¹⁵ PFU/injection depending on the desired result. Low doses of antigen generally induce a strong cell-mediated response, whereas high doses of antigen generally induce an antibody-mediated immune response. Precise amounts of the therapeutic composition also depend on the judgment of the practitioner and are peculiar to each individual.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

### Example 1

### Upstream Cell Culture and Adenovirus Amplification

Characterization and optimization of the adenovirus vector production process using the Wave suspension process and chromatography purification.

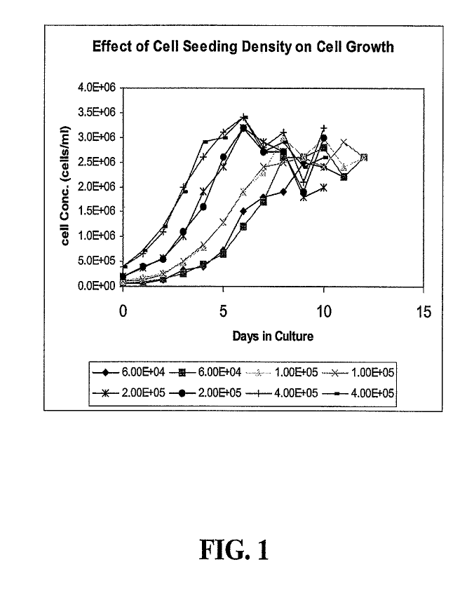

Cell seeding density. 293 suspension cell stock was used to seed shaker flask cultures at various seeding densities. The cultures were placed on top of an orbital shaker (Innova 2000, New Brunswick Scientific, Inc.) set at a shaking speed of 90-100 rpm. Cells were cultured inside an incubator set at 37° C., 10% CO2 and 90% relative humidity. Daily culture samples were taken for cell counting. Data for cell growth is shown FIG. 1. Satisfactory cell growth was achieved with a wide range of cell seeding densities. Longer lag phase was observed at cell seeding densities lower than 1×10⁵ cells/mL. For optimal cell growth the cell seeding density is recommended to be higher than 1×10⁵ cells/mL.

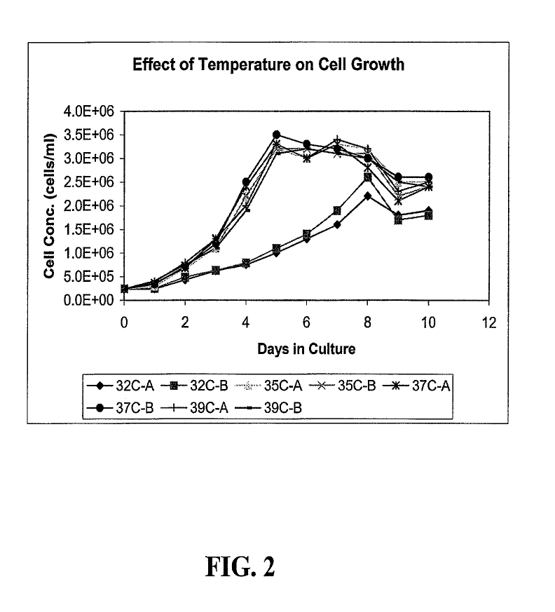

Culture temperature. 293 suspension cell stock maintained in the process development (PD) lab was used to seed shaker flask cultures at a seeding density of 2.4×10⁵ cells/mL. The cultures were placed on top of an orbital shaker (Innova 2000, New Brunswick Scientific, Inc.) set at a shaking speed of 90-100 rpm. Cells were cultured inside incubators set at 32° C., 35° C., 37° C., and 39° C. All incubators were controlled at 10% CO2 and 90% relative humidity. Culture samples were taken for cell counting. Data for cell growth is shown FIG. 2. Satisfactory cell growth was achieved at incubation temperatures of 35° C., 37° C., and 39° C., while significant reduction in cell growth was observed at 32° C. The data suggest that the incubation temperature for growth of 293 suspension cells should be controlled at no less than 35° C. in order to maintain optimal cell growth.

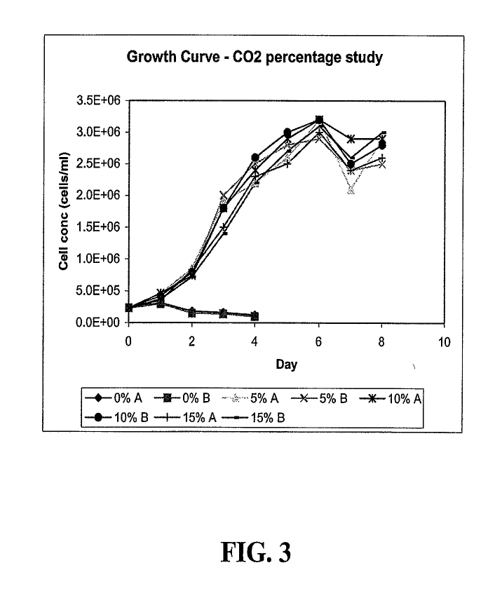

CO₂ percentage. 293 suspension cell stock was used to seed shaker flask cultures at a seeding density of 2.3×10⁵ cells/mL. The cultures were placed on top of an orbital shaker (Innova 2000, New Brunswick Scientific, Inc.) set at a shaking speed of 90-100 rpm. Cells were cultured inside incubators set at 0, 5, 10 and 15% CO2. All incubators were controlled at 37° C. and 90% relative humidity. Daily culture samples were taken for cell counting. Data for cell growth is shown FIG. 3. Satisfactory cell growth was achieved at CO₂ percentages of 5%, 10% and 15%, with almost no cell growth was observed when no CO₂ was provided. The data suggest that growth of 293 suspension cells required CO₂ in the culture environment and should be maintained between 5-15%.

Shaking speed. Due to the formation of foam in the culture media under higher shaking speed, cell culture was optimized. Optimal shaking speed was determined by the lack of foam formation and adequate suspension of the 293 cells. The range was found to be 80-120 rpm.

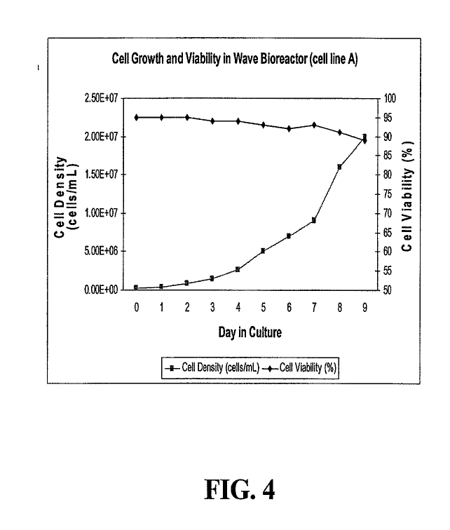

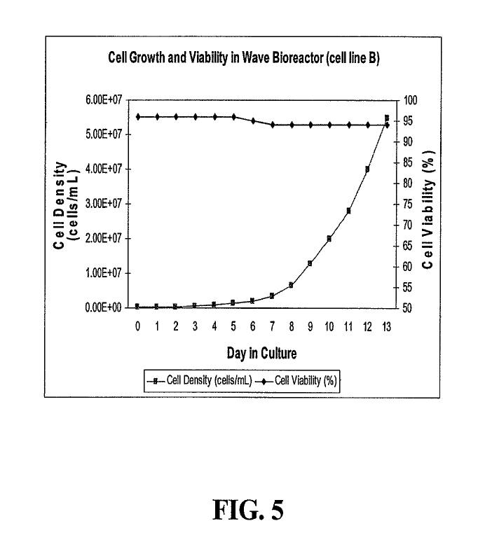

Cell growth in Wave bioreactor 293/HeLa suspension cells. Wave-20 biorector was seeded with 293 or HeLa suspension cells at a cell seeding density of 2×10⁵ cells/mL. Cells were allowed to grow inside the bioreactor. Culture condition was controlled at 36.5° C., pH at 7.20, rocking at 10 rpm. Daily culture sample was taken for cell counting. When cell concentration reached 2×10⁶ cells/mL, media perfusion was initiated to allow further growth of the cells inside the bioreactor. Data for cell growth is shown in FIG. 4 for 293 cells and FIG. 5 for HeLa cells. For 293 suspension cells, cell concentration reached approximately 2×10⁷ cells/mL at the end of the perfusion culture with good cell viability. For HeLa suspension cells, cell concentration reached more than 5×10⁷ cells/mL at the end of the perfusion culture with good cell viability. The cell growth data show that cell culture in the Wave bioreactor can be intensified to reach high cell concentrations by using media perfusion. The high cell concentration is expected to improve the unit productivity of adenovirus vectors.

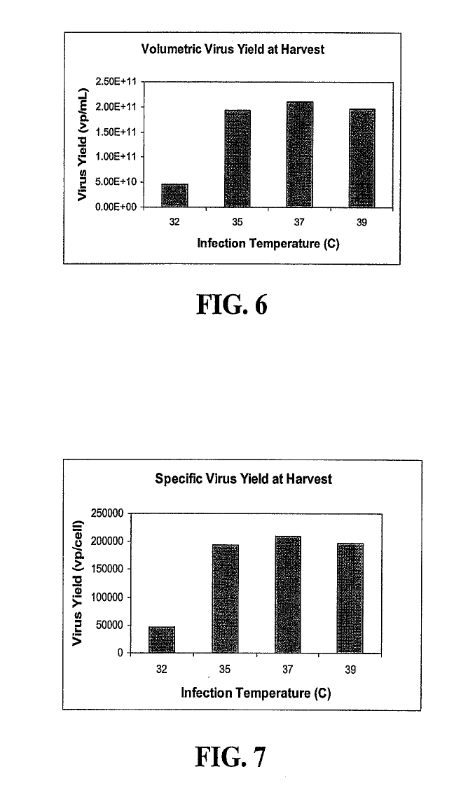

Infection temperature. 293 suspension cells grown in CD293 media were centrifuged and the cell pellet was resuspended in fresh CD293 media at 1×10⁶ cells/mL. The cells were infected with Ad-p53 at MOI of 50 vp/cell in duplicate shaker flask cultures. Infected flasks were placed in incubators set at 32° C., 35° C., 37° C., and 39° C. All incubators were controlled at 10% CO₂ and 90% relative humidity. On day 2 post infection, all flasks were harvested. Sample from each flask was treated with Tween20, Benzonase and filtered using Serum Acrodisc filter (0.2 μm). Virus particle concentration was determined using a HPLC method. Virus yield at different infection temperatures is shown in FIGS. 6 and 7.

Optimal virus production was achieved at 37° C. Significantly lower virus yield was seen at 32° C. Reduced virus production occurred at 35° C. and 39° C., although not significantly. Therefore, 37° C. is recommended for production of adenovirus in 293 suspension cells.

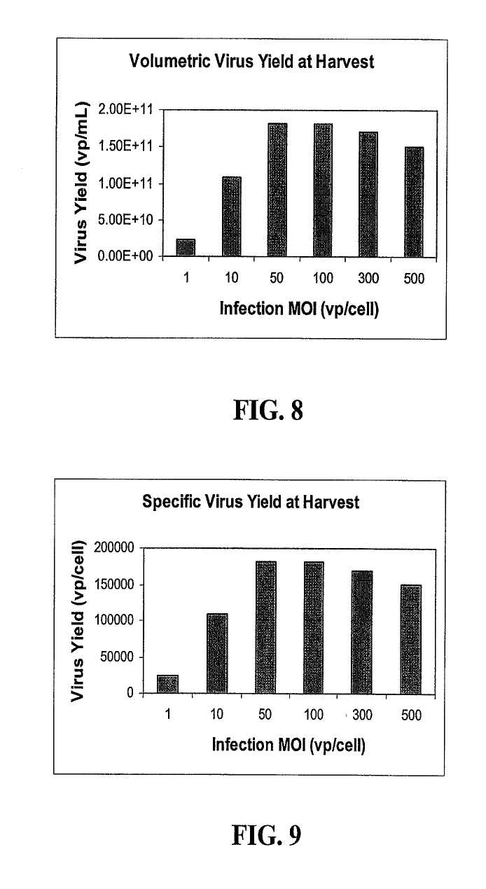

MOI. 293 suspension cells grown in CD293 media were centrifuged and the cell pellet was resuspended in fresh CD293 media at 1×10⁶ cells/mL. The cells were infected with Ad-p53 at MOI of 1, 10, 50, 100, 300, and 500 vp/cell in duplicate shaker flask cultures. Infected flasks were placed in incubators set at 37° C., 10% CO₂ and 90% relative humidity. On day 2 post infection, all flasks were harvested. Sample from each flask was treated with Tween20 and Benzonase, and filtered using Serum Acrodisc filter (0.2 μm). Virus particle concentration was determined using a HPLC method. Virus yield at different infection temperatures is shown in FIGS. 8 and 9.

Relatively consistent virus yield was observed with MOIs at or above 50 vp/cell. Virus production was reduced at MOIs lower than 50 vp/cell. The data indicate that MOIs higher than 100 did not benefit virus production and MOIs between 50-100 vp/cell appear to be the optimal range for adenovirus production in 293 suspension culture.

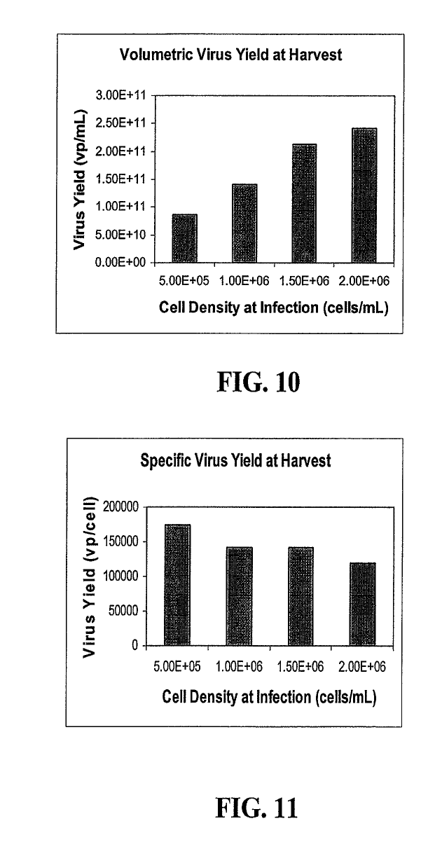

Infection cell density. 293 suspension cells grown in CD293 media were centrifuged and the cell pellet was resuspended in fresh CD293 media at concentrations of 5×10⁵, 1×10⁶, 1.5×10⁶, and 2×10⁶ cells/mL. The cells were infected with Ad-p53 at MOI of 50 vp/cell in duplicate shaker flask cultures. Infected flasks were placed in incubators set at 37° C., 10% CO2 and 90% relative humidity. On day 2 post infection, all flask s were harvested. Sample from each flask was treated with Tween20, treated with Benzonase, and filtered using Serum Acrodisc filter (0.2 μm). Virus particle concentration was determined using a HPLC method. Virus yield at different infection temperatures is shown in FIGS. 10 and 11.

Volumetric virus yield increased with the cell density at infection. However, cell-specific virus yield decreased as the infection cell concentration increased. From a adenovirus manufacture efficiency point of view, maximize volumetric productivity is more important than obtaining high cell-specific productivity. Therefore, cells should be infected at a cell concentration that is as high as possible.

Supplementation of fresh media at virus infection. 293 suspension cells grown in CD293 media were centrifuged. Both the cell pellet and spent media supernatant were retained. The cell pellet was resuspended in the spent media supernatant supplemented with different percentage of fresh CD 293 media at 1×10⁶ cells/mL. Those included,

1. No fresh CD 293 media supplementation (100% spent media)

2. supplemented with 25% fresh CD 293 media

3. supplemented with 50% fresh CD 293 media

4. 100% fresh CD 293 media (no spent media)

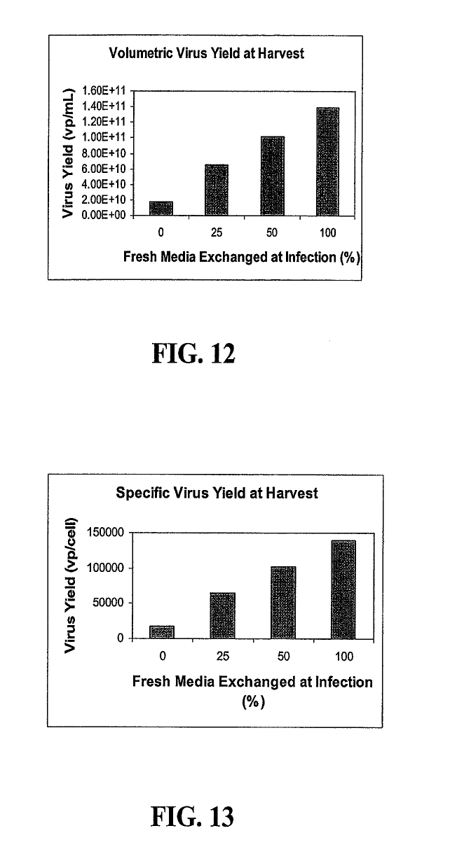

The cells were infected with Ad-p53 at MOI of 50 vp/cell in duplicate shaker flask cultures. Infected flasks were placed in incubators set at 37° C., 10% CO₂ and 90% relative humidity. On day 2 post infection, all flasks were harvested. Sample from each flask was treated with Tween20, treated with Benzonase, and filtered using Serum Acrodisc filter (0.2 μm). Virus particle concentration was determined using a HPLC method. Virus yield at different infection temperatures is shown in FIGS. 12 and 13.

The virus yield data clearly demonstrate that infection of 293 cells in fresh CD 293 media is required in order to achieve high adenovirus production. It is possible that both nutrient limitation and metabolite product inhibition in the spent media contributed to the reduction in the adenovirus production. The data has significant implications for scale up of adenovirus production in 293 suspension culture. A mechanism for large scale media exchange needs to be developed at the time of virus infection. Possible mechanisms include centrifugation, filtration, and fast media perfusion for a shot period of time. The method used at Introgen was to culture cells to a high cell concentration (approximately 1×10⁷ cells/mL) using media perfusion. At the time of virus infection, dilute the concentrated culture with fresh media together with the virus for infection to achieve media exchange without using centrifugation and filtration steps.

### Example 2

### Downstream Processing and Purification

Adenovirus crude lysate was harvested from a Wave-20 bioreactor. The harvest was used for downstream processing and purification studies.

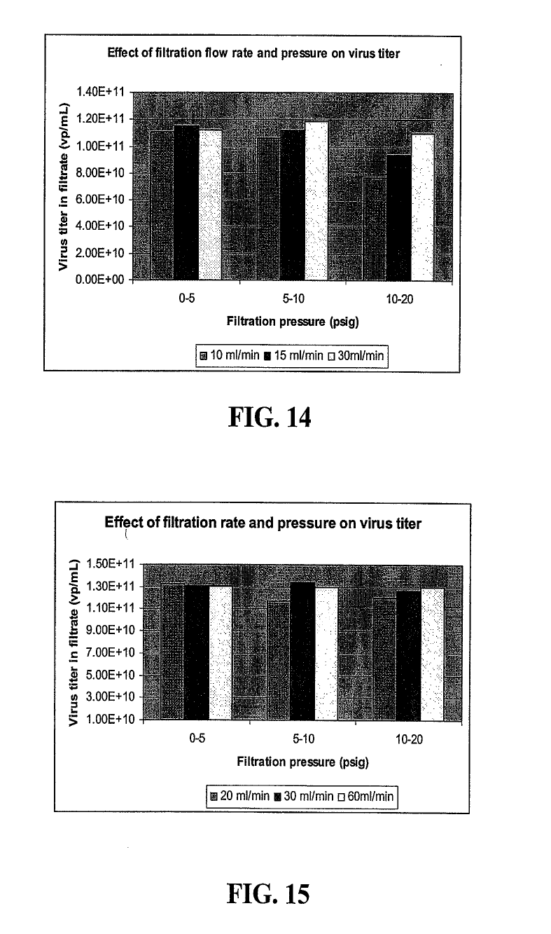

Clarification. A nominal 5.0 μm Optiscale Polygard CN filter (Millipore, Cat # SN50A47FH3, Lot # C3AN31419) and a nominal 0.5 μm Polysep II filter (Millipore, Cat #SGW6A47FH3, Lot # C5AN46927) were used for clarification of the crude virus harvest. The virus harvest was first clarified using the Polygard CN filter. The filtrate collected from the Polygard CN filter was further filtered through the 0.5 μm Polysep II filter. The effect of filtration rate and pressure on virus titer was examined. The result is shown in FIGS. 14 and 15.

Since 2 Polygard CN filters were used in parallel in tandem with 1 Polysep II filter, the filtration rate used for the Polysep II filter was twice that used for the Polygard CN filters. Consistent virus filtration was observed with a wide range of filtration speed and pressure. The combination of 2 5.0 μm Optiscale Polygard CN filters with 1 0.5 μm Polysep II filter appeared to work well for the clarification of crude adenovirus harvest from 293 suspension cultures.

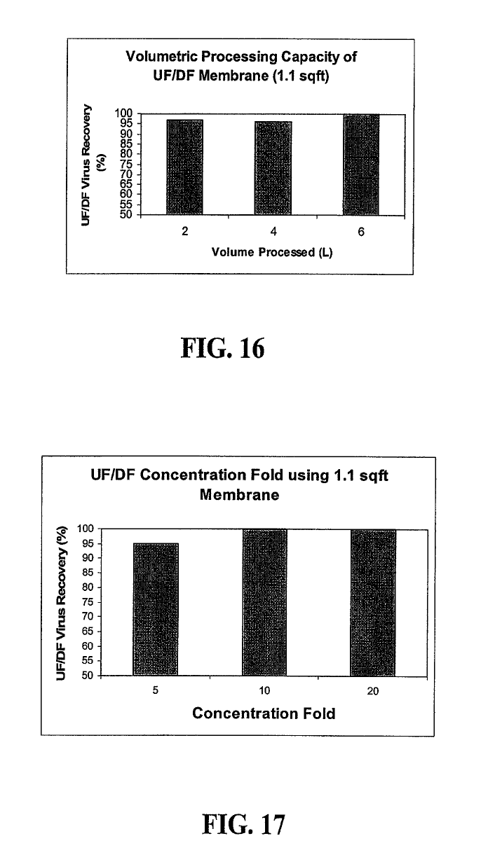

Concentration and diafiltration by tangential flow filtration (UFDF). The clarified virus harvest was concentrated and diafiltered using a 300KD (Millipore Pellicon II, Biomax 300KD membrane) membrane. Process parameters used for the UFDF step were examined with regard to virus recovery. Those included membrane capacity, fold of concentration, and diafiltration efficiency. The result is shown in FIG. 16 and 17. Consistent virus recovery was achieved in a membrane capacity of 2-6 L/1.1ft2. Satisfactory virus recovery was attained at a concentration fold range between 5 to 20-folds. Satisfactory virus recovery was attained with a wide range of feeding flow rates. The feeding flow rates controls the transmembrane pressure of the UFDF process. The data also show that high buffer exchange efficiency was achieved at all the feeding flow rates tested.

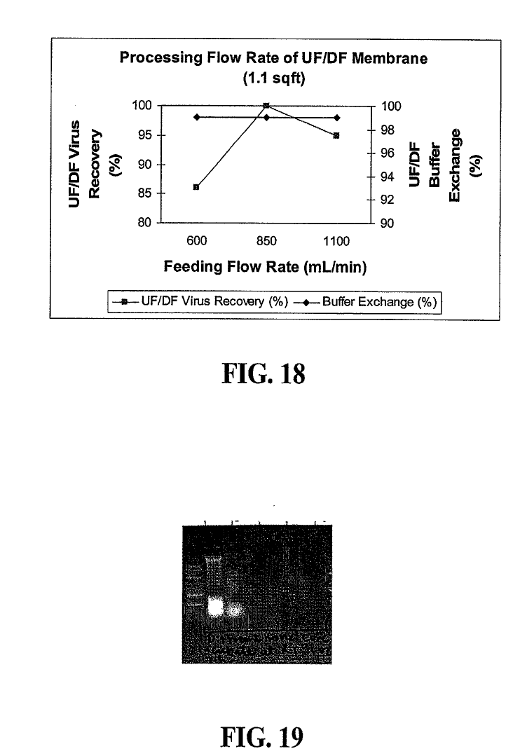

Overall, the study data demonstrate that the tangential flow filtration concentration and diafiltration process is robust and delivers high virus recovery and buffer exchange efficiency. See FIG. 18.

Enzyme treatment step. An endonuclease enzyme (Benzonase) treatment step is included in the adenovirus production process at Introgen to reduce the size of potential nucleic acid impurities in the final vector product. The UFDF virus material is treated with Benzonase at a concentration of 100 u/mL at room temperature for at least 16 hours. To test the efficacy of the Benzonase treatment step, an experiment was performed using different concentrations of Benzonase to treat UFDF processed adenovirus material at room temperature for 1 hour. The treated material was analyzed on a 0.7% agarose gel for the presence of different sized DNA. The result is shown in FIG. 19.

Without Benzonase treatment, significant amount of large sized DNA was seen in the UFDF material (lane labeled as 0 u/mL). Dramatic reduction in the amount and size of DNA was seen with Benzonase treatment. At Benzonase concentrations higher than 50 u/mL, DNA was no longer detectable on the gel after 1 hour treatment at room temperature. The data suggest that the Benzonase treatment step utilized in the adenovirus production process at Introgen is effective at reducing the amount and size of contaminating DNA.

Chromatography purification. The inventors contemplate the demonstration of Source 15Q will have a high resin capacity, and will also function in a wide range of between 5×10¹¹ vp/mL and 3.5×10¹² vp/mL of resin and still produce purified adenovirus of acceptable quality and quantity. A loading density of 2×10¹² vp/mL resin is seen as a useful target value for the anticipated 2-fold scale up.

It is also contemplated that the linear flow rate used for purification will function in a wide range of between 60 and 180 cm/hr and still produce purified adenovirus.

It is further contemplated that the inclusion of a 40 mS/cm hold step during the linear elution gradient will provide for a useful reduction in the amount of residual BSA contaminant while not affect overall yield or any other measure of product quality.

It is still further contemplated that the run pH may vary between 7.5 and 9.0 and will still produce purified adenovirus meeting target specifications.

It is contemplated that a gradient study will demonstrate that a 30 column volume linear gradient volume provides both an acceptable recovery and an acceptable level of residual BSA contaminant, especially when combined with the 40 mS/cm hold step. The useful linear gradient range (in column volumes) could range from 30 to 50 column volumes, with the higher volume gradients resulting in somewhat lower yield by peak broadening.

It is further contemplated that a step gradient study will define the effects of both raising the conductivity of the load and performing the elution in stepwise as opposed to linear fashion. If variation in salt conditions were to occur during a run, this study defines the expected results. As a side benefit, a step gradient could be potentially utilized in future manufacturing processes to produce final product of equivalent quality to that currently made using a linear gradient. Confirmation of equivalent levels of additional residual contaminants would be required before any implementation.

The inventors also contemplate that an anion-exchange chromatography step in an adenoviral purification process may provide a useful amount of viral clearance, approximately 2 logs in the case of two chosen representative viral agents (BVDV and MMV).

It is contemplated that residual DNA contamination will be substantially reduced by the chromatography step to levels acceptable by WHO guidelines of <10 ng (<10,000 pg) residual cellular DNA per dose under all loading conditions.

The subsequent studies will substantially define the chromatography unit operation used and will provide justification for use going forward in scaled-up procedures.

### Example 3

### Liquid Formulations

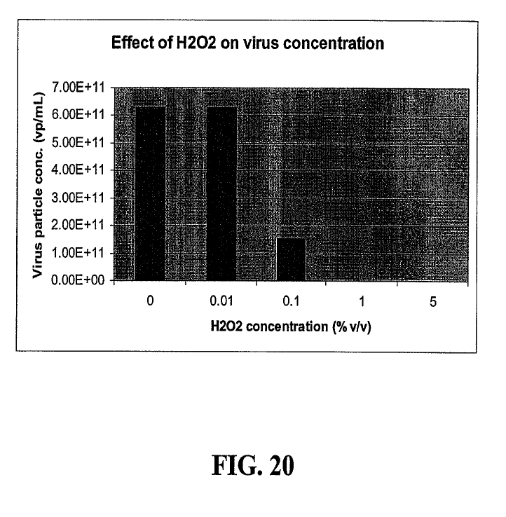

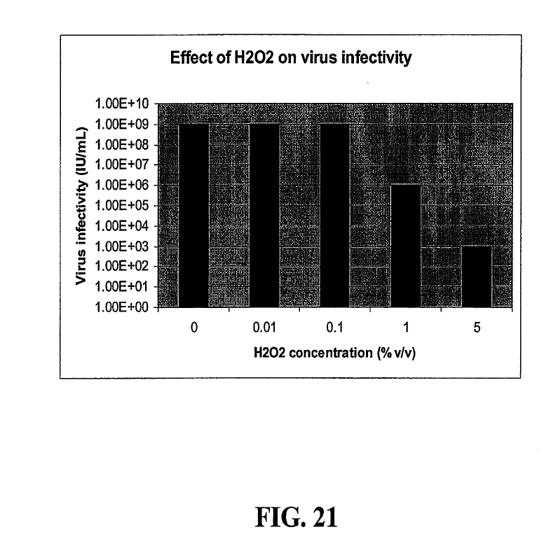

Effect of oxidation on adenovirus. Hydrogen peroxide (H₂O₂) was used as an oxidizer. Different concentrations of H₂O₂ were added to an adenovirus vector preparation at a virus concentration of 6.3×10¹¹ vp/mL. After 1 to 2 hours incubation at room temperature, the samples were analyzed for virus particle concentration and infectivity by a HPLC and a CPE assay, respectively. The data is shown in FIG. 20 and 21.

Significant reductions in virus particle concentration and infectivity were observed at H₂O₂ concentrations higher than 1%. Because of the higher sensitivity of the HPLC assay, reduction in virus particle concentration was seen even at a H₂O₂ concentration of 0.1%. The data show that adenovirus is sensitive to oxidation damage.

Anti-oxidation excipients. Based on the inventors' experience and the literature, ethanol and arginine were evaluated as potential anti-oxidation agents to be used in adenovirus formulations.

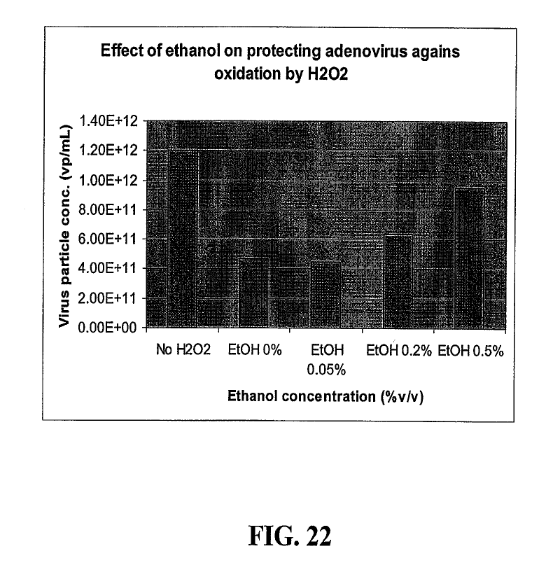

Ethanol Different concentrations of ethanol were added to a adenovirus vector preparation with a virus particle concentration of 1.2×10¹² vp/mL. H₂O₂ was added to each of the preparations to a final concentration of 1% (v/v). After 1.5 hours incubation at room temperature, the samples were analyzed by HPLC for virus particle concentration. The data is shown in FIG. 22.

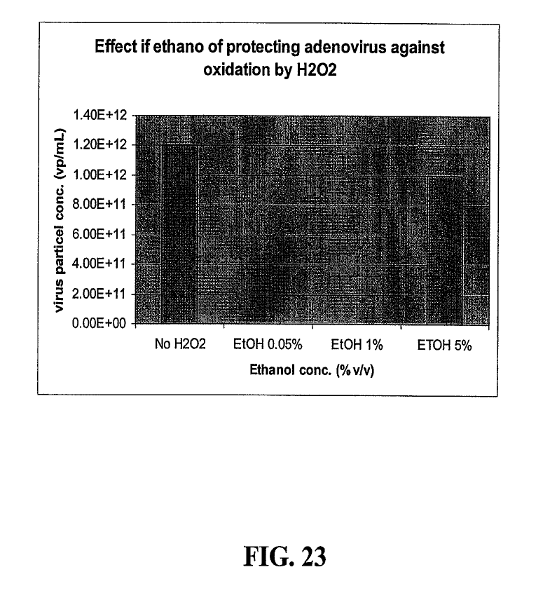

Reduction in virus particle concentration was noticed in the presence of H₂O₂. Addition of ethanol protected the adenovirus against H₂O₂ oxidation damage. Ethanol protection was concentration dependent. Significant protection was seen at 0.5%. To confirm the result, the incubation time was increased to 24 hours and ethanol concentration increased to 5%. Data for virus particle concentration as analyzed by HPLC is shown in FIG. 23.

Increased oxidation damage was observed as the incubation time increased to 24 hours. No protection was evident at ethanol concentration as high as 1%. Satisfactory protection was attained at a higher concentration of 5%.

Overall the data suggest that ethanol is an effective anti-oxidant that could be used to develop formulations for adenovirus.

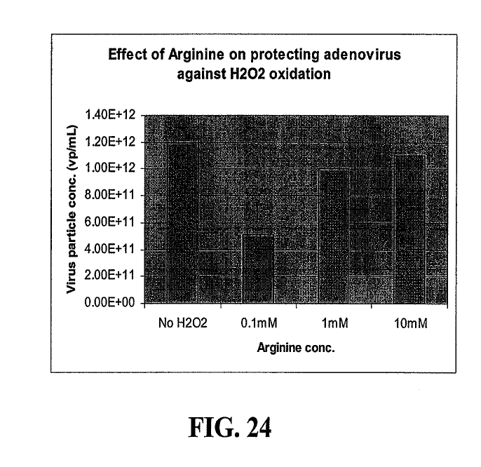

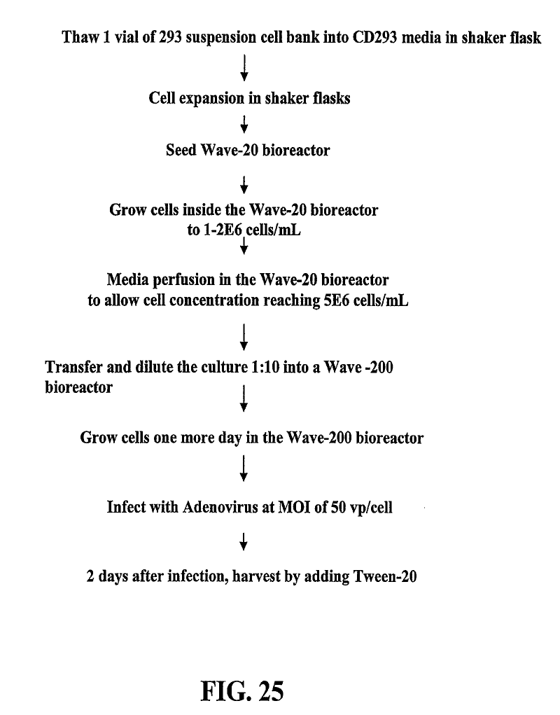

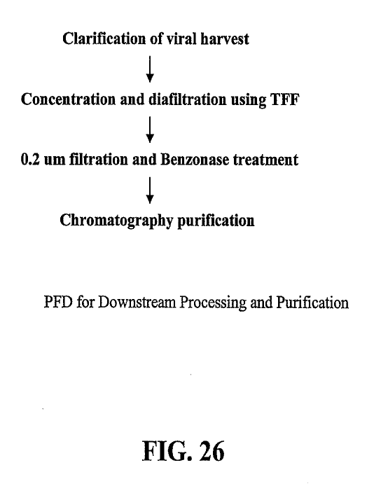

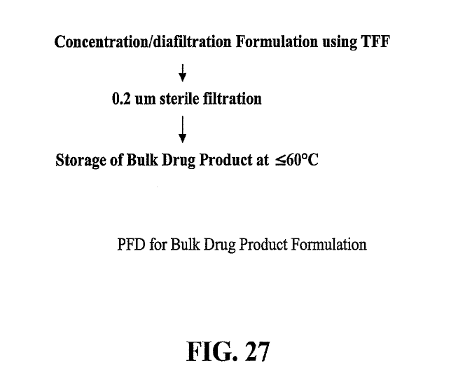

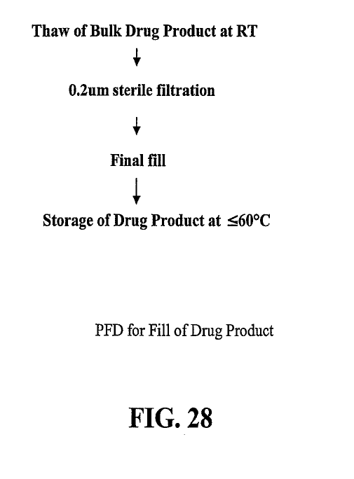

Arginine. In U.S. Pat. No. 6,689,600, the amino acid Arginine as a possible excipient for the formulation of adenovirus. Because of the presence of unsaturated bond in the Arginine molecule, it could be considered as a potential anti-oxidant. Similar studied as stated above for ethanol was carried out with Arginine. Different concentrations of Arginine were added to the adenovirus vector preparation. H₂O₂ was added to each of the preparations to a final concentration of 1% (v/v). After 1.5 hours incubation at room temperature, the samples were analyzed by HPLC for virus particle concentration. The data is shown in FIG. 23 and 24.

Similar to that observed for ethanol, addition of Arginine protected the adenovirus against H₂O₂ oxidation damage. Protection was also concentration dependent. Significant protection was seen at 1 and 10 mM concentrations. However, when the incubation time increased to 24 hours, no protection was observed at either 1 or 10 mM concentrations.

Data from the studies indicate that adenovirus is sensitive to oxidation which is expected to be a factor causing adenovirus instability during long term storage at 4° C. Two potential anti-oxidants, ethanol and Arginine, have been demonstrated to have varying degrees of anti-oxidation effects. Both agents achieved adenovirus protection in the presence of H₂O₂.

The studies include ethanol and Arginine into the following base formulation developed in the previous studies, 20 mM Tris+0.15M NaCl+0.1% Tween-80+0.5% PEG, pH=8.20 Adenovirus will be formulated in those formulations at 1×10¹¹, 2.5×10¹¹, 5×10¹¹, and 1×10¹² vp/mL. The formulated virus will be stored at 4° C. and room temperature for extended period of time. Samples will be taken at different time points for stability assessment.

The purpose of this liquid formulation development project is to develop novel formulation for long term storage of adenovirus vectors in a liquid state at or above refrigeration temperature. Adenoviral vectors used for human gene therapy are routinely stored at ultralow temperatures such as ≦−60° C. to maintain the long term stability of the vector. Ultralow temperature storage is expensive and not convenient for transportation and distribution. Furthermore, ultralow temperature storage is not readily available in some parts of the world and thus limits the use of adenoviral vector product in those areas.

Materials include:

Adenoviral vector: AdCMVp53 (P/N 09-00024, Lot # 003485P)

PEG: (SIGMA Cat# P3640, Lot# 093K0153)

Tween-80 (SIGMA P8074, Lot# 073K00641)

Tris (Angus, Cat# 15-40510, Lot# F8046)

NaCl (Mallikrodt, V47473, Lot# 7713)

Water for Irrigation (WFIr) (B. BraunR5007, Lot# J4J1547)

Methods:

Buffer preparation. The buffer used for formulation was 20 mM Tris+0.1 5M NaCl, pH=8.20. The solution was sterilized by filtering through a 0.2 μm filter unit. PEG was dissolved in WFIr to a concentration of 10% (w/v). The stock solution was sterilized by filtering through a 0.2 μm filter. Tween-80 was dissolved in WFIr to a final concentration of 10% (v/v). The stock solution was sterilized by filtering through a 0.2 μm filter.

Formulation of adenovirus. The AdSCMVpS3 virus was diafiltered into the formulation buffer using tangential flow filtration with a 300KD membrane (Biomax 300KD, Millipore). The virus suspension was sterilized by filtering through a 0.2 μm filter. The virus concentration was 1.0×10¹² vp/mL. The sterilized PEG and Tween-80 stock solutions were added to the virus suspension to the final concentrations as shown in TABLE 2.

The formulated virus suspension was vialed into sterile glass vials at 1 mL per vial. The vials were stoppered and crimped. The vials were grouped and stored at −2020 C., 2-8° C. (refrigerated), and 25° C., respectively, for stability study.

Stability study time points. On the following storage time points, samples were retrieved from storage and tested for stability.

1 week—Two vials from each formulation were retrieved from 25° C. storage and were used for infectivity, viral particle size and HPLC analysis. Vials were also observed visually for presence of gross precipitation.

1 month—Two vials of each formulation were retrieved from all three temperature conditions and were used for infectivity, viral particle size and HPLC analysis. Vials were also observed visually for presence of gross precipitation

3 month—Two vials of each formulation were retrieved from all three temperature conditions and were used for infectivity, viral particle size and HPLC analysis. Vials were also observed visually for presence of gross precipitation.

4 month—Two vials of each formulation were retrieved from all three temperature conditions and were used for infectivity, viral particle size and HPLC analysis. Vials were also observed visually for presence of gross precipitation.

Results:

Results from the different formulations at different time points are shown in Table 3, 4, and 5. In the formulation that did not contain Tween-80 (Formulation A), increase in particle size was observed after 1 month storage. The increase in particle size is believed to have caused the precipitation seen in the vials stored at 2-8° C. and 25° C. After 4 month storage at 25° C., virus infectivity decreased approximately 2 logs. Total virus particle concentration analyzed by HPLC also decreased. For storage temperatures of 2-8° C. and −20° C., similar loss of virus infectivity and virus particle concentration were observed. Therefore, Formulation A will not be used to formulate adenovirus product.

For the formulations that contain Tween-80 (Formulation B and C), virus remained stable after 1 month storage at 25° C. No increases in particle size and virus precipitation were observed. The result suggests that the presence of Tween-80 in the formulation prevented virus precipitation in non-frozen, liquid storage and extended the stability of the adenovirus product. Similar stability data were seen at −20° C. and 2-8° C. storage.

Unfortunately, loss of virus stability was observed at 3 and 4 month storage time points for both Formulation B and C under all three storage temperatures. It appears that most of the decrease in virus infectivity occurred between 3 and 4 month storage. A decrease in virus particle concentration was also noticed by HPLC analysis. The decrease in virus stability is not caused by virus aggregation/precipitation as no appreciable change in virus particle size was observed and no visible precipitation was seen in the container. Possible mechanisms for the loss of virus stability are oxidation, deglycosylation, and deamidation of virus proteins. The fact that PEG and Tween-80, which are prone to contain trace amount of peroxide, are included in the formulations makes oxidation a likely mechanism for the loss of virus infectivity.

For 25° C. storage condition, an increase in the HPLC retention time was seen as the virus titer decreased. It appears that both the infectivity and the HPLC assays are able to detect changes in virus stability during storage, thus are stability indicating assays. On the other hand, results from the particle size assay do not correlate with the stability of the virus and is not a stability indicating assay.

Result from this formulation study indicates that inclusion of Tween-80 in the liquid formulation helped to prevent virus aggregation/precipitation during storage at 2-8° C. and 25° C. In formulations containing Tween-80, virus maintained stability at 25° C. for up to one month at a virus concentration of 1×10¹² vp/mL. Unfortunately, loss of virus stability was observed after 3-4 month storage in all the formulations evaluated. Possible virus degradation mechanisms are proposed and will be examined in future formulation studies.

Evaluation of 20 mM Tris+10% or 20% glycerol, pH8.20 at 3 different Ad titers—Ad-p53 virus was formulated into the following buffers at 3 different concentrations, 1×10¹¹, 5×10¹¹, and 1×10¹² vp/mL. The formulated virus was sterilly filled into glass vials at 1 mL per vial. The vials were sealed and crimped. The vials were divided and stored separately at 4° C. and 25° C. At different storage time points vials were taken for infectivity (CPE assay) and SEC-HPLC (size exclusion HPLC) viral particle determination. The results are shown in Table 8. Form #1: 20 mM Tris+10% glycerol, pH 8.20 and Form #2: 20 mM Tris+20% glycerol, pH 8.20 at a storage temperature 4° C. and 25° C.

TABLE 7
220-037 Evaluation of adenoviral formulation at pH 8.20 at 3 different Ad titers

| | | Storage | CPE titer (IU/mL) | | | | SEC-HPLC titer (vp/mL) | | | |
|---|---|---|---|---|---|---|---|---|---|---|
| Vialing | Testing | time | 25° C. | | 4° C. | | 25° C. | | 4° C. | |
| titer | date | month | Form#1 | Form#2 | Form#1 | Form#2 | Form#1 | Form#2 | Form#1 | Form#2 |
| 1 × 10¹¹ | 22 Aug. 2006 | 0 | 8 × 10⁹ | 8 × 10⁹ | 8 × 10⁹ | 8 × 10⁹ | 1 × 10¹¹ | 1 × 10¹¹ | 1 × 10¹¹ | 1 × 10¹¹ |
| | 20 Sep. 2006 | 1 | 2 × 10¹⁰ | 2 × 10¹⁰ | 1 × 10¹⁰ | 1 × 10⁹ | 1 × 10¹¹ | 9.5 × 10¹⁰ | 1 × 10¹¹ | 9.7 × 10¹⁰ |
| | 31 Oct. 2006 | 2 | 1.10 × 10¹¹ | 1.2 × 10¹¹ | 1.2 × 10¹¹ | 1.2 × 10¹¹ | 1.0 × 10¹⁰ | 1.0 × 10¹⁰ | 1.0 × 10¹⁰ | 1.0 × 10¹⁰ |
| 5 × 10¹¹ | 22 Aug. 2006 | 0 | 4 × 10¹⁰ | 4 × 10¹⁰ | 4 × 10¹⁰ | 4 × 10¹⁰ | 5.3 × 10¹¹ | 5.3 × 10¹¹ | 5.3 × 10¹¹ | 5.3 × 10¹¹ |
| | 20 Sep. 2006 | 1 | 8 × 10¹⁰ | 8 × 10¹⁰ | 4 × 10¹⁰ | 2 × 10¹⁰ | 6 × 10¹¹ | 5.5 × 10¹¹ | 5.9 × 10¹¹ | 5.3 × 10¹¹ |
| | 31 Oct. 2006 | 2 | 6.2 × 10¹¹ | 6.0 × 10¹¹ | 6.1 × 10¹¹ | 5.9 × 10¹¹ | 8.0 × 10¹⁰ | 4.0 × 10¹⁰ | 8.0 × 10¹⁰ | 4.0 × 10¹⁰ |
| 1 × 10¹² | 22 Aug. 2006 | 0 | 8 × 10¹⁰ | 8 × 10¹⁰ | 8 × 10¹⁰ | 8 × 10¹⁰ | 1.1 × 10¹² | 1 × 10¹² | 1.1 × 10¹² | 1 × 10¹² |
| | 20 Sep. 2006 | 1 | 8 × 10¹⁰ | 4 × 10¹⁰ | 4 × 10¹⁰ | 4 × 10¹⁰ | 1.1 × 10¹² | 1.1 × 10¹² | 1.1 × 10¹² | 1 × 10¹² |
| | 31 Oct. 2006 | 2 | 1.2 × 10¹² | 1.2 × 10¹² | 1.3 × 10¹² | 1.1 × 10¹² | 8.0 × 10¹⁰ | 8.0 × 10¹⁰ | 8.0 × 10¹⁰ | 4.0 × 10¹⁰ |

### Example 4

### Improved Concentration and Diafiltration by Tangential Flow Filtration

Introduction. Tangential Flow Filtration (UFDF) has been used and disclosed by others (including Introgen's previous patent applications and issued patents) for the concentration and diafiltration of adenovirus. However, in those cases, the UFDF step was used mainly for the purpose of virus concentration and exchange the spent media to a buffer suitable for treatment of the virus particle suspension with Benzonase (a broad spectrum nuclease) and subsequent anionic exchange chromatography. The UFDF step was not intended as the sole virus purification step since significant contaminants were still present after diafiltratoin for adenvirus produced in culture media containing serum.

Accordingly, the inventors have experimented with using UFDF as the primary adenovirus purification method. Towards this end, Adenoviral vector is harvested from the cell culture media and clarified using microfiltration to remove large cellular debris. The clarified virus harvest may then be subsequently concentrated. Optionally, the harvested virus may also be treated with Benzonaze (a broad spectrum endonuclease) in order to digest large free nucleic acids present in the harvest. Following concentration the virus concentrate is purified by diafiltration using UFDF through porous membranes having molecular weight cutoff in the range of 300-1000. By using the formulation buffer as the diafiltration buffer, the purified virus may also formulated during the diafiltration purification process. This may benefit the production process by simplification.

Virus Production and Clarification. 293 suspension cells were grown in CD293 media in a Wave bioreactor. Cells were grown to a cell density 8.0×10⁵ cells/ml. Total volume of the bioreactor at the time of infection was 100 L. The cells were infected with Ad-pmda7 at MOI of 100 vp/cell. Two days post infection, 1 L of Tween-20 was added to the bioreactor. Three days post infection all cells were harvested and a subjected to clarification. HPLC analysis of the clarified 100 L sample was performed. Results were compared to subsequent results following concentration and diafiltration by tangential flow filtration.

Concentration and diafiltration by tangential flow filtration (UFDF). The clarified virus harvest was concentrated and diafiltered using a 500KD (Millipore Pellicon II, Biomax 500KD membrane) membrane. Process parameters used for the UFDF step were examined with regard to virus recovery. Those included virus titer (vp/ml), fold of concentration, HPLC purity, recovery percentage and total virus yield. The result is shown in Table 8. Diafiltration was carried out up to 60× and samples were collected at 10×, 20×, 30×, 40× and 60×. UFDF inlet feeding was set at 10 psi.

TABLE 8
Results of UFDF on Virus Recovery
and Yield With a 500KD Membrane

| | | HPLC | | |
|---|---|---|---|---|
| | Titer (vp/ml) | Purity | Recovery | Total Yield (vp) |
| Clarified Harvest | 1.20 × 10¹¹ | 5.3% | NA | 1.20 × 10¹⁶ |
| 10-fold UFDF | 2.30 × 10¹² | 78.6% | 90 | 1.08 × 10¹⁶ |
| 20-fold UFDF | 2.20 × 10¹² | 89.5% | 89 | 1.07 × 10¹⁶ |
| 30-fold UFDF | 2.30 × 10¹² | 93.5% | 89 | 1.06 × 10¹⁶ |
| 40-fold UFDF | 1.80 × 10¹² | 97.1% | 90 | 1.08 × 10¹⁶ |
| 60-fold UFDF | 1.50 × 10¹² | 98.5% | 79 | 9.50 × 10¹⁵ |

### REFERENCES

The following references, to the extent that they provide exemplary procedural or other details supplementary to those set forth herein, are specifically incorporated herein by reference.
U.S. Pat. No. 4,352,883
U.S. Pat. No. 5,670,488
U.S. Pat. No. 5,824,544
U.S. Pat. No. 5,932,210
U.S. Pat. No. 6,194,191
U.S. Pat. No. 6,514,943
U.S. Pat. No. 6,689,600
U.S. Pat. No. 6,726,907
U.S. Patent Publn. 20020182723
U.S. Patent Publn. 20030232035
U.S. Patent Publn. 20040033239
U.S. Patent Publn. 20040106184
U.S. Patent Publn. 20040229335
U.S. Patent Publn. 20050158283
U.S. Patent Publn. 20330229354
U.S. Patent Ser. No. 60/026,667
U.S. Patent Ser. No. 60/203,078
U.S. Patent Ser. No. 60/406,591
Aboud et al., Arch. Virol., 71:185-195, 1982.
Bahnemann et al., Abs. Pap. ACS, 180:5. 1980.
Baichwal and Sugden, In. Gene Transfer, Kucherlapati (Ed.), NY, Plenum Press, 117-148, 1986.
Batra et al., Am. J. Respir. Cell Mol. Biol., 21(2):238-45, 1999.
Berg, Biotechniques, 14(6):972-978., 1993.
Bett, Proc. Natl. Acad. Sci. USA, 91(19):8802-8806, 1994.
Blackwell et al., Arch. Otolaryngol. Head. Neck Surg., 125(8):856-863, 1999.
Brett et al., J. Immunol., 150:2869-2884, 1993.
Chen and Okayama, Mol. Cell Biol., 7(8):2745-2752, 1987.
Chillon et al., J. Virol., 73(3):2537-2540, 1999.
Chillon et al., J. Virol., 73(3):2537-40, 1999.
Chroboczek et al., Virology, 186:280-285, 1992.
Coffin, In: Virology, Fields et al. (Eds.), Raven Press, NY, 1437-1500, 1990.
Coupar et al., Gene, 68:1-10, 1988.
Cristiano et al., Cancer Detect. Prev., 22(5):445-454, 1998.
Crooks et al., J. Chromatogr., 502(1):59-68, 1990.
Dorai et al., Int. J. Cancer, 82(6):846-52, 1999.
Fechheimer, et al., Proc Natl. Acad. Sci. USA, 84:8463-8467, 1987.
Feldman et al., Cardiovasc. Res., 32(2):194-207, 1996.
Fraley et al., Proc. Natl. Acad. Sci. USA, 76:3348-3352, 1979.
Gamier et al., Cytotechnology, 15(1-3):145-155, 1994.
Gilbert, Williamsburg BioProcessing Conference, Nov. 18-21, 1996.
Golasten et al, New Engl. J. Med., 309(11983):288-296, 1983.
Gopal, Mol. Cell Biol., 5:1188-1190, 1985.
Graham and Prevec, In: Methods in Molecular Biology. Gene Transfer and Expression Protocol, Murray (Ed.), Humana Press, Clifton, N.J., 7:109-128, 1991.
Graham and Prevec, Mol. Biotechnol., 3(3):207-220, 1995.
Graham and Van Der Eb Harland and Weintraub, 1985
Graham et al., J. Gen. Virl., 36(1):59-74, 1977.
Graham, J. Gen. Virol., 68(Pt 3):937-940, 1987.
Griffiths, J. Histochem. Cytochem., 34(11):1389-1398, 1986.
Han et al., Biol. Pharm. Bull., 22(8):836-40, 1999.
Hearing and Shenk, J. Molec. Biology, 167:809-822, 1983.
Hearing et al., J. Virology, 67:2555-2558, 1987.
Hermens and Verhaagen, Prog. Neurobiol., 55(4):399-432, 1998.
Hermonat and Muzycska, Proc. Natl. Acad. Sci. USA, 81:6466-6470, 1984.
Hurwitz et al., Hum. Gene Ther., 10:441-48, 1999.
Huyghe et al., Human Gene Therapy, 6:1403-1416,1996.
Irie et al., Antisense Nucleic Acid Drug Dev., 9(4):341-9, 1999.
Ishibashi et al, J. Clin. Invest., 92:883-893, 1993.
Ishibashi et al, J. Clin. Invest., 93:1885-1893, 1994.
Jones and Shenk, Cell, 13:181-188, 1978.
Kaneda et al., Science, 243:375-378, 1989.
Kato et al, J. Biol. Chem., 266:3361-3364, 1991.
Klein et al., Nature, 327:70-73, 1987.
Larsson and Litwin, Dev. Biol. Standard., 66:385-390, 1987.
Lesch, Biol. Psychiatry, 45(3):247-53, 1999.
Mann et al., Cell, 33:153-159, 1983.
Marienfeld et al., Gene Ther., 6(6):1101-13, 1999.
McGrath et al., J. Virol., 25:923-927, 1978.
Mincheff et al., Eur. Urol., 38(2):208-17, 2000.
Mizrahi, Dev. Biol. Stand., 55:219-230, 1983.
Morris et al., Environ. Mol. Mutagen., 27(1):10-8, 1996.
Morrison et al., J. Gen. Virol., 78(Pt 4):873-8, 1997.
Morrison et al., J. Gen. Virol., 78(Pt 4):873-878, 1997.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nilsson and Mosbach, Dev. Biol. Standard., 66:183-193,
O'Neil and Balkovic, Biotechnology, 11 (2):173-178, 1993.
Parks et al., J. Virol., 71(4):3293-8, 1997.
PCT Appl. WO 94/17178
PCT Appl. WO 98/00524
PCT Appln. WO 00/32754
PCT Appln. WO 99/41416
Perrin, Vaccine, 13(13):1244-1250, 1995.
Petricciani, Dev. Biol. Standard., 66:3-12, 1985.
Petrof, Eur. Respir. J., 11(2):492-7, 1998.
Phillips et al., In: Large Scale Mammalian Cell Culture (Feder and Tolbert, eds.), Academic Press, FL, 1985.
Potter et al., Proc. Natl. Acad. Sci. USA, 81:7161-7165, 1984.
Reddy et al., Virology, 251(2):414-26, 1998.
Reddy et al., Virology, 251(2):414-26, 1998.
Ridgeway, In: Vectors: A Survey of Molecular Cloning Vectors and Their Uses, Rodriguez et al. (Eds.), Stoneham: Butterworth, 467-492, 1988.
Rippe, et al., Mol. Cell Biol., 10:689-695, 1990.
Robbins and Ghivizzani, Pharmacol Ther, 80(1):35-47, 1998.
Robbins et al., Trends Biotechnol., 16(1):35-40, 1998.
Smith and Lee, Anal Biochem., 86(1):252-263, 1978.
Stewart et al., Gene Ther., 6(3):350-63, 1999.
Tanzawa et al, FEBS Letters, 11 8(1):81-84, 1980.
Temin, In: Gene Transfer, Kucherlapati (Ed.), NY, Plenum Press, 149-188, 1986.
Tibbetts, Cell, 12:243-249, 1977.
Tur-Kaspa et al., Mol. Cell Biol., 6:716-718, 1986.
van Wezel, Nature, 216:64-65, 1967.
Vanderkwaak and Alvarez, Curr. Opin. Obstet. Gynecol., 11(1):29-34, 1999.
Wadell et al, Ann. NY Acad. Sci., 354:16-42, 1980.
Wagner et al., Science, 260:1510-1513, 1993.
Wang et al., In: Animal Cell Technology: Basic and Applied Aspects, Kaminogawa et al., (eds), 5:463-469, Kluwer Academic Publishers, Netherlands, 1993.
Wang et al., Cytotechnology, 9:41-49, 1992.
Wang et al., Proc. Japan. Soc. Animal Cell Tech., 1994.
Watanabe, Atherosclerosis, 36:261-268, 1986.
Wilson, J. Clin. Invest., 98(11):2435, 1996.
Wilson, Nature, 365:691-692, 1993.
Wong et al., Gene, 10:87-94, 1980.
Wu and Wu, Biochemistry, 27: 887-892, 1988.
Wu and Wu, J. Biol. Chem., 262:4429-4432, 1987.
Yang and Russell, Proc. Natl. Acad. Sci. USA, 87:4144-4148, 1990.
Yotnda et al., Gene Ther., 8:930-37, 2001.
Zheng et al., J. Gen. Virol., 80(Pt 7):1735-42, 1999.

1. A method of producing a purified adenovirus composition comprising:
(a) inoculating a growth medium to an initial population of host cells of at least 1×10⁴ cells/ml;
(b) growing host cells in a disposable bioreactor having the medium at a culture temperature of about 35° C. to about 40° C. in an atmosphere of about 1% to 20% CO₂ at a shaking speed of about 50 to 150 rpm;
(c) providing nutrients to the host cells by perfusing the cells with a media containing glucose at a concentration of 0.5 to 5 g/L;
(d) infecting the host cells at a cell density of at least 1×10⁵ cells/mL with an adenovirus at an infection temperature of about 35° C. to 40° C. at an MOI of about 50 to 100 MOI, wherein 25% to about 100% of the growth medium is exchange prior to or during administration of the adenovirus to the host cells;
(e) lysing the host cells to provide a cell lysate comprising adenovirus; and
purifying adenovirus from the lysate by ion exchange chromatography and size partitioning purification;
wherein the purified adenovirus composition has a purity of less than 10 ng of contaminating DNA per 1×10¹² viral particles.

2. The method of claim 1, wherein the host cells are grown in a bioreactor.

3. The method of claim 2, wherein the bioreactor is a bag bioreactor with a volume of 1 L to 1000 L.

4. The method of claim 1, wherein the host cells are grown at a culture temperature of about 37° C.

5. The method of claim 1, wherein the host cells are grown at CO₂ percentage of about 10%.

6. The method of claim 1, wherein the host cells are grown at a shaking speed of about 80 to 120 rpm.

7. The method of claim 1, wherein the host cells are infected at a temperature of about 37° C.

8. The method of claim 1, wherein the host cells are infected when at a cell density of at least 1×10⁶ cells/ml.

9. The method of claim 1, wherein about 25% to about 50% of the medium is exchanged prior or concurrently with infection.

10. The method of claim 1, wherein about 100% of the medium is exchanged prior or concurrently with infection.

11. The method of claim 1, wherein in purifying adenovirus from the lysate is by size partitioning purification using a dialysis membrane, a porous filter, or a tangential flow filtration device.

12. The method of claim 11 wherein the size partitioning membrane has a pore size of less than about 0.08 microns and greater than about 0.0001 microns.

13. The method of claim 11, wherein filtration rate is a circulating speed of 500-1500 mL/min/fsf2 and the filtration pressure is within the range of 1-20 psig.

14. The method of claim 11, wherein size partitioning purification is by tangential flow filtration.

15. The method of claim 14, wherein membrane capacity is about 2 L/1.1 ft² to about 6 L/1.1 ft².

16. The method of claim 14, wherein concentration fold was in the range of 5-fold to 20-fold.

17. The method of claim 14, wherein feeding flow rate is 500 ml/min to 1500 ml/min.

18. The method of claim 1, wherein the virus is purified to a pharmaceutically acceptable degree without the use of ion exchange chromatography.

19. The method of claim 1, wherein the host cells are grown in a perfusion chamber, a bioreactor, a flexible bed platform or by fed batch.

20. The method of claim 1, wherein the purified adenovirus composition has a purity of less than 5 ng of contaminating DNA per 1 milliliter dose.

21. The method of claim 1, wherein the adenovirus comprises an adenoviral vector encoding an exogenous gene construct.

22. The method of claim 1, wherein the cell lysate is treated with an endonuclease.

23. The method of claim 1, wherein the cells are grown as a suspension culture.

24. The method of claim 1, wherein the cells are grown as an anchorage-dependent culture.

25. The method of claim 1, wherein at least 5×10¹⁵ to 1×10⁶ viral particles are obtained from a single culture preparation.

26. A virus formulation comprising:
(a) a purified virus at a concentration of at least 1×10⁵ vp/mL; and
(b) an anti-oxidant.

27. The formulation of claim 26, wherein the antioxidant is ethanol, arginine, or both ethanol and arginine.

28. The formulation of claim 27, wherein ethanol is present in a concentration of at least 0.5% to 10% v/v

29. The formulation of claim 27, wherein arginine is present in a concentration of at least 0.5 to 15 mM.
